SEQUENCE LISTING (7 sequences)

SEQ ID NO: 1 - protein, 10 aa, synthetic construct
GRRRRELDPM

SEQ ID NO: 2 - protein, 9 aa, synthetic construct
RQRRELDPM

SEQ ID NO: 3 - protein, 8 aa, synthetic construct
KIEAVKSE

SEQ ID NO: 4 - protein, 8 aa, synthetic construct
KIEAVQSE

SEQ ID NO: 5 - protein, 8 aa, synthetic construct
VGCGVLLS

SEQ ID NO: 6 - protein, 7 aa, synthetic construct
GCGVLLS

SEQ ID NO: 7 - protein, 12 aa, synthetic construct
  VARIANT 3..4: note = X can be any amino acid
  VARIANT 6..7: note = X can be any amino acid
  VARIANT 9..10: note = X can be any amino acid
HEXXHXXGXXHD

(19) United States
(12) Patent Application Publication
(10) Pub. No.: US 2025/0017963 A1
(43) Pub. Date: Jan. 16, 2025

(54) SYNTHETIC CYTOKINE CIRCUITS TO PROMOTE INFILTRATION AND CLEARANCE OF IMMUNE EXCLUDED SOLID TUMORS BY ENGINEERED IMMUNE CELLS

(71) Applicant: The Regents of the University of California, Oakland, CA (US)

(72) Inventors: Wendell A. LIM, San Francisco, CA (US); Gregory ALLEN, San Francisco, CA (US)

(21) Appl. No.: 18/708,921

(22) PCT Filed: Nov. 22, 2022
(86) PCT No.: PCT/US2022/080370

§ 371 Date: May 9, 2024

Related U.S. Application Data
(60) Provisional application No. 63/286,984, filed on Dec. 7, 2021.

(51) Int. Cl. A61K 35/17; A61K 39/00; C07K 14/55; C07K 14/725; C07K 16/30; C12N 5/0783

(52) U.S. Cl. CPC A61K 35/17; A61K 39/4611; A61K 39/4631; A61K 39/464468; A61K 39/464488; C07K 14/55; C07K 14/7051; C07K 16/3069; C12N 5/0636; A61K 2239/13; C07K 2319/03; C07K 2319/50

(57) ABSTRACT

Provided herein, among other things, is a cytotoxic immune cell (e.g., a cytotoxic T cell) that expresses an engineered immune receptor (such as a CAR or TCR) whose cytotoxicity within the tumor microenvironment is enhanced by a pro-inflammatory protein that is induced only when the cell binds to either a tissue-specific or cancer-associated antigen. As such, the present cells can be used for the treatment of cancers that are associated with solid tumors.

### CROSS-REFERENCING

This application claims the benefit of U.S. provisional application Ser. No. 63/286,984, filed on Dec. 7, 2021, which application is incorporated by reference in its entirety.

### STATEMENT REGARDING FEDERALLY SPONSORED RESEARCH

This invention was made with government support under grant no U54 CA244438 awarded by The National Institutes of Health. The government has certain rights in the invention.

### INCORPORATION BY REFERENCE OF SEQUENCE LISTING PROVIDED AS AN XML FILE

A Sequence Listing is provided herewith as a Sequence Listing XML, “UCSF-661WO_SEQ_LIST” created on Nov. 21, 2022 and having a size of 9 kilobytes. The contents of the Sequence Listing XML are incorporated by reference herein in their entirety.

### INTRODUCTION

Despite the remarkable success of anti-CD19 Chimeric Antigen Receptor (CAR) T cells in treating B cell cancers the application of CAR T cells to solid tumors has proven far more difficult. There are two significant challenges in developing CAR T cells for solid tumors. The first challenge is identifying truly tumor-specific cell surface antigens to target. The second challenge is to overcome the immunosuppressive microenvironment found in solid tumors.

With respect to the first challenge, there do not appear to be ideal single antigen targets: attacking most tumor associated antigens leads to severe adverse effects associated with cross-reaction with normal organs that also express the antigen. Even the FDA approved CD19 CAR T cells have “on-target/off-tumor” killing—in this case the destruction of normal B cells. While the B cell compartment is expendable, such toxicity would be unacceptable for CAR T cells that cross-react with epithelial organs that are indispensable and cannot be eliminated.

Current strategies for targeting solid tumors primarily focus on tumor-associated antigens which are over-expressed in tumors compared to normal tissue—for example HER2 (ERBB2). Unfortunately, attempts to target tumor-associated antigens have been plagued by “on-target/off-tumor” toxicities due CAR T cells acting against normal tissue that express tumor-associated antigens at a low level. In the case of HER2 “on-target/off-tumor” toxicity led to unexpected and fatal pulmonary edema. Follow up HER2 targeted CAR T cell trials with a lower potency CAR T cell product (decreased dose, scFv affinity) have had limited efficacy. Similar dose limiting “on-target/off-tumor” toxicities have also been seen in clinical trials of CAR T cells targeting CEACAM5, EGFR, Mesothelin and CA9 due to pulmonary, intestinal or biliary tract toxicity. In addition, several other promising CAR T cell products have had to be abandoned or weakened in pre-clinical development due to excessive “on-target/off-tumor” toxicity in murine models, including those targeting the antigens SSEA4, B7-H4, GD2, and others.

With respect to the second challenge, it is well established that immunosuppressive cells are recruited into the tumor microenvironment. For example, solid tumors often contain a variety of different immunosuppressive cells, including a specialized subset of CD4⁺ T cells called regulatory T cells or Tregs. Tregs are highly immunosuppressive and play a crucial role in maintaining immune tolerance during homeostasis and suppressing exacerbated immune responses in various pathological conditions. These cells have been shown to suppress the anti-tumor immune response and promote tumor growth. As such, solid tumors often suppress the desired effects of cytotoxic T cells that target such tumors.

Many current efforts in T cell engineering are focused on trying to increase the potency and durability of CAR T cells to overcome suppressive solid tumor microenvironments. However, if such increases in CAR T potency are not coupled with an improved ability to kill only tumor cells, then the adverse effects caused by off-tumor cross-reactivities may become even more severe and lethal.

Thus, there is a great need for next generation cellular therapies to more precisely distinguish tumor tissue from normal tissue and overcome the tumor microenvironment, for the treatment of solid tumors.

### SUMMARY

The present disclosure provides a cytotoxic immune cell (e.g., a cytotoxic T cell) that expresses an engineered immune receptor (such as a CAR or TCR) whose cytotoxicity within the tumor microenvironment is enhanced by a pro-inflammatory protein that is induced only when the cell binds to either a tissue-specific or cancer-associated antigen. As such, the present cells can be used for the treatment of cancers that are associated with solid tumors. In some embodiments, the engineered immune cell may comprise the following components: (a) a nucleic acid encoding an immune receptor (e.g., a CAR or TCR) that is activated by binding to a cancer-associated antigen in a solid tumor; (b) a binding triggered transcriptional switch (BTTS) that is independently activated (i.e., independently from the immune receptor) by either a tissue- or a cancer-associated antigen in the solid tumor; and (c) a nucleic acid encoding a pro-inflammatory protein. In these cells, binding of the immune receptor to the cancer-associated antigen activates the immune cell and binding of the BTTS to its antigen activates expression of the pro-inflammatory protein, and, optionally, the immune receptor if the immune receptor is not constitutively expressed in the cell, where the expression of the pro-inflammatory protein is “local” to the immune cell in the tumor microenvironment, and not systemic. In these embodiments, expression of the pro-inflammatory protein helps overcome the immunosuppressive environment that typically exists in solid tumors and, in addition, helps the immune cells infiltrate immune excluded tumors, thereby enhancing the ability of the cells to kill cancer cells.

In any embodiment, the pro-inflammatory protein is a cytokine selected from IL-2, IL-12, IL-15, IL-7, CD40L, or a non-natural variant of IL-2, IL-12, IL-15, IL-7, CD40L that has pro-inflammatory activity, an anti-PD1 antibody, an anti-PDL1 antibody, a decoy resistant IL-18 or a dominant negative TGF-β, for example. In any embodiment, the pro-inflammatory protein is a cytokine may be IL-2. The cancer-associated antigen recognized by the immune receptor depends on the cancer that is being targeted and, in some embodiments, may be selected from an antigen listed in Table 1. Likewise, the antigen recognized by the BTTS will depend on the cancer that is being targeted and may either be tissue-specific (i.e., specific for the tissue from which the malignant cells are derived, e.g., a lung-specific antigen for lung cancer, etc.) or selected from may be selected from an antigen listed in Table 1. The antigens recognized by the immune receptor and the BTTS may be the same or different. As will be explained in greater detail below, the cells may be used to treat lung cancer, colorectal cancer, pancreatic cancer, prostate cancer, liver and biliary tract cancers, bladder cancer, brain cancer (e.g., GBM), esophageal cancer, ovarian cancer, kidney cancer, melanoma, gastric/stomach cancer, breast cancer, mesothelioma, uterine, testicular, and head and neck (including thyroid), among others.

Examples of such cells and their use are described in further detail below.

### BRIEF DESCRIPTION OF THE DRAWINGS

FIG. 1: KPC tumor cell expressing human CD19 and firefly luciferase were engrafted into the tail of the pancreas of immunocompetent C57/B16 mice and treated 9 days later with 2e6 engineered mouse CD3+ T cells by tail vein injection. Tumor control was only seen with aMeso CAR T cells that were also engineered with an anti-CD19 SynNotch driving expression of mouse IL-2. Plots show tumor burden as measured by average +/−S.E.M. of luciferase signal from abdominal cavity and overall survival (n=4-5 per group). 150 mg/kg cytoxan was given as preconditioning on day 8 to all mice

FIG. 2: KPC tumor cell expressing human CD19 were engrafted sub-cutaneously into C57/B16 mice and treated 7 days later with 2e6 engineered mouse CD3+ T cells by tail vein injection. Tumor control was only seen with aMeso CAR T cells that were also engineered with an anti-CD19 SynNotch driving expression of mouse IL-2. Plots show tumor burden as measured by average (thick line) and individual measurements of tumor by caliper measurements and overall survival (n=4-5 per group). 150 mg/kg cytoxan was given as preconditioning on day 6 to all mice.

FIG. 3: B16F10 OVA tumor cell expressing human CD19 were engrafted sub-cutaneously into immunocompetent C57/B16 mice and treated 9 days later with 2e6 engineered mouse CD3+ T cells by tail vein injection. Tumor control was only seen with aMeso CAR T cells that were also engineered with an anti-CD19 SynNotch driving expression of mouse IL-2. Plots show tumor burden as measured by average +/−S.E.M. of tumor size by caliper measurements and overall survival (n=4-5 per group). 150 mg/kg cytoxan was given as preconditioning on day 8 to all mice.

FIG. 4: KPC tumor cell expressing human CD19 were engrafted sub-cutaneously into C57/B16 mice and treated 9 days later with 2e6 engineered mouse CD3+ T cells by tail vein injection. Tumors were collected from mice 14 days after T cell treatment and analyzed by IHC for CD3 infiltration. In mice treated with aMeso CAR T cells (top row) T cells are found predominantly at the tumor periphery. In mice treated with aMeso CAR T cells+cytokine circuit (aCD19SN=>IL2, bottom row, T cell infiltrate extensively within the tumor. Representative images are shown. 150 mg/kg cytoxan was given as preconditioning on day 8 to all mice.

FIG. 5. KPC tumor cell expressing human CD19 were engrafted sub-cutaneously into C57/B16 mice and treated 6 days later with 1e6 engineered mouse CD3+ T cells by tail vein injection. For each graph we see no T cell group (black) and treatment group. (top) red indicates aMeso CAR T cells only, (2^{nd}) brown indicates aMeso CAR T cells with expression of IL2 from a constitutive promoter, (3^{rd}) green indicates expression of IL2 from an NFAT promoter, (4^{th}) blue, indicates IL2 production from an anti-CD19 synNotch. Plots show tumor burden as measured by average (thick line) and individual measurements of tumor by caliper measurements and overall survival (n=4-5 per group). 150 mg/kg cytoxan was given as preconditioning on day 5 to all mice.

FIG. 6. KPC tumor cell expressing human CD19 were engrafted sub-cutaneously into C57/B16 mice and treated 9 days later with 2e6 engineered mouse CD3+ T cells by tail vein injection. Mean+/−standard deviation of tumor size by caliper measurements is shown. We see that in untreated mice (black) and mice treated with standard anti-meso CAR (blue) tumors grown out. In mice treated with an anti-FAP SynNotch driving aMeso CAR and IL-2 complete tumor control is achieved. Overall survival is also depicted above (n=4-5 per group). 150 mg/kg cytoxan was given as preconditioning on day 5 to all mice.

FIG. 7. KPC tumor cell expressing human CD19 were engrafted sub-cutaneously into C57/B16 mice and treated 8 days later with 2e6 engineered mouse CD3+ T cells by tail vein injection. Mean+/−standard deviation of tumor size by caliper measurements is shown. We see that in untreated mice (green) and mice treated with anti-meso CAR T cells expressing the ortho2 receptor (blue) tumors grow out. In mice treated with anti-Meso CAR T cells expressing the ortho2R beta chain and an anti-CD19 SynNotch driving ortho-IL2 clone 1G12 significant tumor control is achieved. (n=4-5 per group). 150 mg/kg cytoxan was given as preconditioning on day 7 to all mice.

FIGS. 8A-F show that synthetic synNotch→IL-2 circuits can drive local T cell proliferation independent of TCR activation or cooperatively with T cell killing.

FIG. 8A: The tumor microenvironment (TME) acts to suppress T cell activation, including inflammatory cytokine (e.g. IL-2) production. To bypass suppression, we propose to engineer synthetic IL-2 circuits trigged by tumor antigens in a manner independent from TCR/CAR activation.

FIG. 8B: Synthetic IL-2 circuits were created in human primary T cells using anti-CD19 synNotch receptors to drive production of an inflammatory cytokine (super IL-2/sIL-2). IL-2 is produced only when stimulated by A375 tumor cells bearing the cognate CD19 antigen.

FIG. 8C: Synthetic IL-2 circuit drives autocrine proliferation of primary human T cells in vitro, only when the circuit is triggered (here myc-tagged synNotch is activated by anti-myc antibody coated beads).

FIG. 8D: Synthetic IL-2 circuit signals in a paracrine fashion to stimulate proliferation of a bystander population of human T cells that lack a synthetic circuit in vitro. For C and D, median is plotted; shading shows S.E.M., n=3 and filled markers indicate significant expansion >1, right-tailed students t-test, p<0.05.

FIG. 8E: Dual flank tumor model in NSG mice to monitor T cell trafficking in vivo. Primary human T cells were engineered with synthetic anti-CD19→sIL-2 circuit and eff-luc (to track cells) and administered to mice engrafted with CD19⁺ (right) and CD19⁻ (left) K562 tumors. Example bioluminescence imaging shown 7 days after T cell injection. Circles indicate tumors (blue, white) and spleen (red). Plot shows quantification of T cell luminescence over time for CD19⁺ and CD19 tumors. Dashed line shows T cells in CD19⁺ tumor with no circuit added; shading shows S.E.M.

FIG. 8F: Tumor reactive T cells, such as ones bearing an anti-NY-ESO TCR, fail to produce effective cytokine and killing responses against antigen positive tumors. We hypothesize that simultaneously engaging the TCR and a synthetic IL-2 circuit could enhance a local T cell response. In this case T cells bearing an anti-NY-ESO TCR and an anti-membrane-bound GFP (mGFP) synNotch→sIL-2 circuit could function as an AND gate that requires two antigen inputs to stimulate tumor killing allowing more precise recognition strategies. Here a two-flank A375 tumor model in NSG mice, with NY-ESO only on left and NY-ESO/GFP on right was generated. Plots show tumor growth over time. Both autocrine and paracrine forms of the TCR+anti-GFP synNotch→sIL-2 cells show significantly enhanced control of only the dual antigen tumor. Error shading: S.E.M. Dashed line indicates dual antigen tumor growth curve with no T cell treatment.

FIGS. 9A-9E show that autocrine synthetic IL-2 circuits strongly improve T cell cytotoxicity against multiple models of immune-excluded syngeneic tumors.

FIG. 9A: The synthetic IL-2 circuit was recapitulated in mouse T cells producing mouse IL-2 (mIL-2) to test circuits in presence of an intact immune system, suppressive TME and native IL-2 consumer cells.

FIG. 9B: KPC CD19⁺ pancreatic tumors were engrafted subcutaneously into immunocompetent C57/B16 mice and treated 9 days later with synthetic IL-2 circuit T cells and anti-Mesothelin CAR T cells as a two-cell paracrine system. No tumor control was observed in this paracrine configuration, even though KPC tumors express mesothelin.

FIG. 9C: KPC CD19⁺ pancreatic tumors were engrafted as in B and treated 9 days later with T cells engineered with both a synthetic IL-2 circuit and an anti-Mesothelin CAR (autocrine configuration). Significant improvement in tumor control was observed (red lines) compared to anti-Mesothelin CAR T cells combined with dummy synthetic cytokine circuit (synNotch only produces BFP, black lines).

FIG. 9D: KPC CD19⁺ pancreatic tumors were engrafted orthotopically in the pancreas tail and treated 9 days later with engineered T cells. 100% survival was observed only with the addition of the IL-2 circuit out to 120 days (duration of study).

FIG. 9E: B16F10 OVA CD19⁺ melanoma tumors were engrafted orthotopically into immunocompetent C57/B16 mice and treated 8 days later with 2e6 engineered mouse CD8+OT-1 (anti-OVA) T cells. Tumor control was only observed in mice treated with T cells expressing the IL-2 circuit. For B-E All plots show tumor burden measured by average+/−S.E.M. of caliper or bioluminescence measurements and overall survival (n=4-5 per group, *=significant difference in survival with addition of IL-2 circuit using log-rank test, p<0.05).

FIGS. 10A-E show that synthetic Notch based cytokine delivery is required for effective control of KPC tumors.

In these figures, KPC CD19⁺ pancreatic tumors were engrafted subcutaneously into immunocompetent C57/B16 mice and treated 9 days later with T cells as labeled. Plotted is schematic for IL-2 production as well as overall survival for each cell design compared to matched untreated mice. n=4,5 per group.

FIG. 10A: 1e6 anti-Mesothelin CAR T cells with no additional IL-2.

FIG. 10B: 2e6 anti-Mesothelin CAR T cells with systemic IL-2 administered at high dose (250,000 to 750,000 IU/mL) twice daily intraperitoneally for 7 days.

FIG. 10C: 1e6 anti-Mesothelin CAR T cells engineered to constitutively express mIL-2 using a PGK promoter.

FIG. 10D: 1e6 anti-Mesothelin CAR T cells engineered to inducibly express mIL-2 under the control of a NFAT promoter.

FIG. 10E: 1e6 anti-Mesothelin CAR T cells engineered to inducibly express mIL-2 under the control of an anti-CD19 synNotch.

FIG. 11 shows that synthetic IL-2 circuit enables T cell infiltration into immune excluded tumors. KPC CD19⁺ tumors were engrafted subcutaneously, treated with engineered T cells, and analyzed by IHC for T cell infiltration (anti-CD3 stain). Anti-mesothelin CAR T cells (top) failed to penetrate into the tumor, infiltrating the tumor edges (black arrows). Addition of synthetic autocrine IL-2 circuit (bottom) resulted in dramatically increased T cell infiltration into tumor core. Tumors were collected 23 days (left) and 8 days (center) after T cell injection. Zoomed out scale bars are 500 microns, zoomed in are 50 microns.

FIGS. 12A-C: profiling of tumor micro-environment shows expansion and activation of CAR T cells with autocrine IL-2 circuit.

FIG. 12A: Treated KPC CD19⁺ tumors were collected as in (A) after 9 days for analysis by CyTOF using CD45.1 as a marker of adoptively transferred T cells and CD45.2 as marker of native T cells. Native T cells and Regulatory T cells (Tregs) showed expansion in tumors treated with anti-mesothelin CAR+synthetic IL-2 circuit in autocrine or paracrine configuration, while adoptive (CAR) T cells showed far more dramatic expansion only with anti-mesothelin CAR+synthetic IL-2 circuit in autocrine configuration. n=3 samples per treatment, no p value calculated. Counts are normalized to tumor weight.

FIG. 12B: Unsupervised analysis of CyTOF data. UMAP shown for KPC tumors treated by anti-mesothelin CAR+/−IL-2 circuit (autocrine). Labelled numbers indicate clusters by Phenograph. Enrichment was only seen in adoptively transferred CAR T cells when the synthetic IL-2 circuit was engaged,

FIG. 12C: Analysis of tumor infiltrating lymphocytes in markers in CAR T cells (CD45.1) from CyTOF data shows that CAR T cells with the synthetic IL-2 circuits in autocrine show higher expression of markers of IL-2 signaling (pSTAT5), activation (CD25), effector function (Granzyme B) and proliferation (Ki67), while showing decreased expression of markers of exhaustion (Tim3, Lag3, PD1). Matched analysis of native T cells (CD45.2) shows limited IL-2 signaling, activation, effector responses, proliferation, or exhaustion markers, with or without addition of synthetic IL-2 circuit. Mean+/−S.D. is plotted.

FIGS. 13A-D show bypassing tumor immune suppression mechanisms with a synthetic IL-2 delivery circuit

FIG. 13A: Standard CAR/TCR T cell activity in suppressive microenvironments is limited by inhibition of T cell activation, minimal production of IL-2, and consumption of IL-2 by competing native cells (sinks). Activation of both TCR and cytokine signaling, required for the full T cell response (AND gate), is blocked at these steps.

FIG. 13B: Creating a bypass channel for IL-2 production that is independent of CAR/TCR activation can overcome key suppressive steps. New circuits allow initiation of T cell activation via synergistic TCR/cytokine stimulation, leading to positive feedback, T cell activation, proliferation, and efficient killing of tumor cells. The synthetic circuit reconstitutes the key requirements for a strong T cell response in a manner that bypasses key suppressive bottlenecks.

FIG. 13C: Schematic differences between autocrine and paracrine IL-2 signaling in the presence of IL-2 consumers. An autocrine IL-2 circuit provides preferential spatial access to self-made IL-2 in comparison to a paracrine IL-2 circuit, where CAR T cells must compete with other IL-2 consumers (Tregs or T-naive cells).

FIG. 13D: An autocrine IL-2 leads to preferential expansion of IL-2 producers (through T cell activation and upregulation of CD25) in contrast in a paracrine circuit IL-2 producers compete on equal or lesser footing with IL-2 consumers and are not selectively enriched limiting total IL-2 produced and failing to accumulate enough IL-2 to overcome thresholds required for T cell activation.

### DEFINITIONS

As used herein, the terms “treatment,” “treating.” “treat” and the like, refer to obtaining a desired pharmacologic and/or physiologic effect and/or a response related to the treatment. The effect can be prophylactic in terms of completely or partially preventing a disease or symptom thereof and/or can be therapeutic in terms of a partial or complete cure for a disease and/or adverse effect attributable to the disease. “Treatment.” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent (including biologic agents, such as cells), or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject.” “host.” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

The term “refractory”, used herein, refers to a disease or condition that does not respond to treatment. With regard to cancer, “refractory cancer”, as used herein, refers to cancer that does not respond to treatment. A refractory cancer may be resistant at the beginning of treatment or it may become resistant during treatment. Refractory cancer may also called resistant cancer.

The term “histology” and “histological” as used herein generally refers to microscopic analysis of the cellular anatomy and/or morphology of cells obtained from a multicellular organism including but not limited to plants and animals.

The term “cytology” and “cytological” as used herein generally refers to a subclass of histology that includes the microscopic analysis of individual cells, dissociated cells, loose cells, clusters of cells, etc. Cells of a cytological sample may be cells in or obtained from one or more bodily fluids or cells obtained from a tissue that have been dissociated into a liquid cellular sample.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains.

The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional heterodimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5 (215): 215ra172; Glienke et al. Front Pharmacol (2015) 6:21; Kakarla & Gottschalk 52 Cancer J (2014) 20 (2): 151-5; Riddell et al. Cancer J (2014) 20 (2): 141-4; Pegram et al. Cancer J (2014) 20 (2): 127-33; Cheadle et al. Immunol Rev (2014) 257 (1): 91-106; Barrett et al. Annu Rev Med (2014) 65:333-47; Sadelain et al. Cancer Discov (2013) 3 (4): 388-98; Cartellieri et al., J Biomed Biotechnol (2010) 956304; the disclosures of which are incorporated herein by reference in their entirety. Useful CARs also include the anti-CD19-4-1BB-CD35 CAR expressed by lentivirus loaded CTL019 (Tisagenlecleucel-T) CAR-T cells as commercialized by Novartis (Basel, Switzerland).

The terms “T cell receptor” and “TCR” are used interchangeably and will generally refer to a molecule found on the surface of T cells, or T lymphocytes, that is responsible for recognizing fragments of antigen as peptides bound to major histocompatibility complex (MHC) molecules. The TCR complex is a disulfide-linked membrane-anchored heterodimeric protein normally consisting of the highly variable alpha (a) and beta (B) chains expressed as part of a complex with CD3 chain molecules. Many native TCRs exist in heterodimeric αβ or γδ forms. The complete endogenous TCR complex in heterodimeric αβ form includes eight chains, namely an alpha chain (referred to herein as TCRα or TCR alpha), beta chain (referred to herein as TCRβ or TCR beta), delta chain, gamma chain, two epsilon chains and two zeta chains. In some instance, a TCR is generally referred to by reference to only the TCRα and TCRβ chains, however, as the assembled TCR complex may associate with endogenous delta, gamma, epsilon and/or zeta chains an ordinary skilled artisan will readily understand that reference to a TCR as present in a cell membrane may include reference to the fully or partially assembled TCR complex as appropriate.

Recombinant or engineered individual TCR chains and TCR complexes have been developed. References to the use of a TCR in a therapeutic context may refer to individual recombinant TCR chains. As such, engineered TCRs may include individual modified TCRα or modified TCRβ chains as well as single chain TCRs that include modified and/or unmodified TCRα and TCRβ chains that are joined into a single polypeptide by way of a linking polypeptide.

As used herein, the term “binding-triggered transcriptional switch” or “BTTS” refers to any polypeptide or complex of the same that is capably of transducing a specific binding event on the outside of the cell (e.g. binding of an extracellular domain of the BTTS) to activation of a recombinant promoter within the nucleus of the cell. Many BTTSs work by releasing a transcription factor that activates the promoter. In these embodiments, the BTTS is made up of one or more polypeptides that undergo proteolytic cleavage upon binding to the antigen to release a gene expression regulator that activates the recombinant promoter. For example, a BTTS may comprise: (i) an extracellular domain comprising the antigen-binding region of an antigen-specific antibody, wherein this region engages with an antigen on another cell; (ii) a transmembrane domain; (iii) an intracellular domain comprising a transcriptional activator; and (iv) one or more proteolytic cleavage sites (e.g., a masked recognition site for an ADAM protease that between the antigen-binding region and the transmembrane domain of the protein, and a site in the transmembrane that is recognized by γ-secretase); where binding of the antigen binding region to the antigen on another cell induces cleavage at the one or more proteolytic cleavage sites, thereby releasing the transcriptional activator. The released transcriptional activator, in turn, activates expression of a downstream protein. A BTTS can be based on synNotch, A2, MESA, or force receptor, for example, although others are known or could be constructed. As such, a BTTS may comprise one or more protease cleavage sites and an intracellular domain comprising a transcriptional activator, wherein binding of the BTTS to the tissue- or a cancer-associated antigen on another cell causes the BTTS to be cleaved at the protease cleavage site, thereby releasing the transcriptional activator, and wherein the released transcriptional activator induces expression of the pro-inflammatory protein. In some embodiments, a SNIPR (Zhu et al 2021 bioRxiv) may be used.

A “biological sample” encompasses a variety of sample types obtained from an individual or a population of individuals and can be used in various ways, including e.g., the isolation of cells or biological molecules, diagnostic assays, etc. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by mixing or pooling of individual samples, treatment with reagents, solubilization, or enrichment for certain components, such as cells, polynucleotides, polypeptides, etc. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples (e.g., biopsy samples), and cellular samples. Accordingly, biological samples may be cellular samples or acellular samples.

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, nanobodies, single-domain antibodies, and fusion proteins comprising an antigen-binding portion of an antibody and a non-antibody protein.

“Antibody fragments” comprise a portion of an intact antibody, for example, the antigen binding or variable region of the intact antibody. Examples of antibody fragments include Fab, Fab′, F(ab′) 2, and Fv fragments; diabodies; linear antibodies (Zapata et al., Protein Eng. 8 (10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′) 2 fragment that has two antigen combining sites and is still capable of cross-linking antigen.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M. e.g., binding with an affinity of 10⁻⁶ M, 10⁻⁵ M, 10⁻⁴ M, etc.

A “orthogonal” or “orthogonalized” member or members of a binding pair are modified from their original or wild-type forms such that the orthogonal pair specifically bind one another but do not specifically or substantially bind the non-modified or wild-type components of the pair. Any binding partner/specific binding pair may be orthogonalized, including but not limited to e.g., those binding partner/specific binding pairs described herein.

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208.

The terms “synthetic”, “chimeric” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “vector” or “expression vector” is a replicon, such as plasmid, phage, virus, or cosmid, to which another DNA segment, i.e. an “insert”, may be attached so as to bring about the replication of the attached segment in a cell.

The term “heterologous”, as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

The term “cancer specific” refers to protein that is over-expressed on the surface of cancer cells.

The term “activates” in the context of activating expression of the pro-inflammatory protein, means inducing the transcription, translation and secretion of the pro-inflammatory cytokine.

The term “cancer-associated” refers to an antigen that is expressed in cancerous cells but not significantly non-cancerous cells of the same type. Some cancer-associated antigens are expressed on cancer cells and in normal tissues. For example, MSLN is considered a cancer-associated antigen since it is aberrantly expressed various cancer cells (e.g., lung cancers (adenocarcinoma and squamous carcinoma), ovary, peritoneum, endometrium, pancreas, stomach and colon, etc.) but it is also expressed on normal mesothelial cells in the pleura, pericardium, and peritoneum and in epithelial cells on the surface of the ovary, tunica vaginalis, rete testis, and fallopian tubes in trace amounts.

The term “activates” or “activated by” in the context of a CAR or BTTS, means that the CAR or BBTS is activated by binding to one or more antigens on another cell or to multiple different antigens on different cells, where the antigens may be selected from Table 1.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the cell” includes reference to one or more cells and equivalents thereof known to those skilled in the art, and so forth. For instance, an immune receptor that is activated by binding to “a” cancer-associated antigen in a solid tumor could be activated by binding to a single target antigen or to two target antigens (in the case of a tandem CAR and the like).

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As summarized above, the present disclosure provides a cytotoxic immune cell whose cytotoxicity within the tumor microenvironment is enhanced by enhanced by local expression of a pro-inflammatory protein. As noted above, in some embodiments, the engineered immune cell may comprise the following components: (a) a nucleic acid encoding an immune receptor (e.g., a CAR or TCR) that is activated by binding to a cancer-associated antigen in a solid tumor; (b) a binding triggered transcriptional switch (BTTS) that is independently activated (i.e., independently from the immune receptor) by either a tissue- or a cancer-associated antigen in the solid tumor; and (c) a nucleic acid encoding a pro-inflammatory protein. In these cells, binding of the immune receptor to the cancer-associated antigen activates the immune cell and binding of the BTTS to its antigen activates expression of the pro-inflammatory protein and, optionally, the immune receptor if the immune receptor is not constitutively expressed in the cell, where the expression of the pro-inflammatory protein is “local” to the immune cell in the tumor microenvironment.

Cancer-associated antigens in solid tumors to which the CAR and BTTS may bind are listed in Table 1 below.

TABLE 1

| | | |
|---|---|---|
| Mesothelin | Trop2 (TACSTD2) | KREMEN2 |
| FAP | MUC1 | GRIN2D |
| EGFRvIII | ROR1 | CELSR3 |
| IL13RA2 | Claudin18.2 | KISS1R |
| EPHA2 | GPC3 | CDH6 |
| PSMA (FOLH1) | CA9 | TREM1 |
| HER2 | EPCAM | MC1R |
| EGFR | FOLR1 | LIFR |
| PSCA | L1CAM | PRR7 |
| ALPPL2 | CD133 (PROM1) | PTPRN |
| GD2 (B4GALNT1) | CEA (CEACAM5) | CACNG7 |
| BCAN | MUC16 | TRPM1 |
| MOG | PDL1 (CD274) | |
| CSPG5 | ROR2 | |
| CD70 | VEGFR2 (KDR) | |
| MET | CD44v6 | |
| AXL | NKG2D (KLRK1) | |
| MCAM | MLANA (MART1) | |
| DLL3, DLL4 | NY-ESO | |
| nectin4 | TYRP-1 | |
| nectin2 | AFP | |
| nectin3 | MAGE family* | |
| nectin1 | WT-1 | |
| ALK | | |

*Reference to “MAGE family” includes any of the MAGE family members listed in Table 2 of Weon et al (Curr Opin Cell Biol. 2015 37: 1-8), particularly MAGE A1, MAGE A2, MAGE A3, MAGE A4, which are each associated with various solid tumors, e.g., NSCLC, melanoma, breast, ovarian and colon.

### Cells

The cells employed herein are immune cells that contain one or more of the described nucleic acids, expression vectors, etc., encoding the desired components. Immune cells of the present disclosure include mammalian immune cells including, e.g., those that are genetically modified to produce the components of a circuit of the present disclosure or to which a nucleic acid, as described above, has been otherwise introduced. In some instances, the subject immune cells have been transduced with one or more nucleic acids and/or expression vectors to express one or more components of a circuit of the present disclosure.

Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or progenitor thereof, obtained from an individual. As another example, the cell is a cytotoxic cell, or progenitor thereof, obtained from an individual.

Such cells include, e.g., lymphoid cells, i.e., lymphocytes (T cells, B cells, natural killer (NK) cells), and myeloid-derived cells (neutrophil, eosinophil, basophil, monocyte, macrophage, dendritic cells). “T cell” includes all types of immune cells expressing CD3 including T-helper cells (CD4+ cells) and cytotoxic T-cells (CD8+ cells). A “cytotoxic cell” includes CD8+ T cells, natural-killer (NK) cells, and neutrophils, which cells are capable of mediating cytotoxicity responses.

Immune cells encoding a circuit of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, a primary T lymphocyte, a stem cell, or an NK cell is obtained from an individual; and the cell obtained from the individual is modified to express components of a circuit of the present disclosure.

### Binding-Triggered Transcriptional Switches

The BTTS is a cleavable fusion protein contains: (a) an extracellular binding domain comprising a protein binding domain (e.g., scFv or nanobody) that binds to an antigen on another cell, (b) a force sensing region, (c) a transmembrane domain, (d) one or more force-dependent cleavage sites that are cleaved when the force sensing region is activated, and (e) an intracellular domain comprising a transcriptional activator, where binding of the binding domain to the antigen on the surface of a cell induces proteolytic cleavage of the one or more force-dependent cleavage sites to release the transcriptional activator.

In this switch, the fusion protein is cleaved to release the intracellular domain when the extracellular domain of the fusion protein engages with an antigen on another cell. As such, in many cases, the fusion protein will contain a force sensing region (which is typically in the extracellular domain) and one or more force-dependent cleavage sites that are cleaved when the force sensing region is activated. The position of the force-dependent cleavage sites may vary and, in some embodiments the fusion protein may contain at least two cleavage sites. In some cases, one of the cleavage sites may be extracellular and the other may be in the transmembrane domain or within 10 amino acids of the transmembrane domain in the intracellular domain. In any embodiment, the force sensing region and/or the one or more force-dependent cleavage sites may be from a Delta/Serrate/Lag2 (DSL) superfamily protein, as reviewed by Pintar et al (Biology Direct 2007 2:1-13). For example, the force sensing region and/or the one or more force-dependent cleavage sites may be from Notch (see Morsut Cell. 2016 164:780-91), von Willebrand Factor (vWF), amyloid-beta, CD16, CD44, Delta, a cadherin, an ephrin-type receptor or ephrin ligand, a protocadherin, a filamin, a synthetic E cadherin, interleukin-1 receptor type 2 (ILIR2), major prion protein (PrP), a neuregulin or an adhesion-GPCR. Several other examples of this type of protein are known and listed in Pintar, supra. Many members of this family appear to share a similar architecture a region that unfolds and opens up a protease cleavage site (e.g., EGF-like repeats; see Cordle et al Nat. Struct. Mol. Biol. 2008 15:849-857), a trans-membrane segment, and a relatively short (˜100-150 amino acids) intracellular domain. These sequences permit the binding-triggered release of a transcriptional activator from the membrane in their natural environment and can be readily adapted herein.

In some cases, the one or more ligand-inducible proteolytic cleavage sites are selected from S1, S2, and S3 proteolytic cleavage sites. In some cases, the S1 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence. The S3 proteolytic cleavage site is in the transmembrane domain. In many cases, the shear force generated by binding of the extracellular domain of this fusion protein to another cells unfolds the force sensing region (which, in the case of Notch contains EGF-like repeats whereas in other protein is made up of other sequences such as the A2 domain in vWF (see, e.g., J Thromb Hacmost. 2009 7:2096-105, Lippok Biophys J. 2016 110:545-54, Lynch Blood. 2014 123:2585-92, Crawley, Blood. 2011 118:3212-21 and Xy J Biol Chem. 2013 288:6317-24) or modified A2 domain that has, e.g., the R1597W, E1638K and I1628T substitutions. The architecture of such proteins is described in, e.g., Morsut et al, Cell. 2016 164:780-91, WO2016138034 and WO2019099689, among other places).

In some cases, the fusion protein includes an S1 ligand-inducible proteolytic cleavage site. An S1 ligand-inducible proteolytic cleavage site can be located between the HD-N segment and the HD-C segment. In some cases, the S1 ligand-inducible proteolytic cleavage site is a furin-like protease cleavage site. A furin-like protease cleavage site can have the canonical sequence Arg-X-(Arg/Lys)-Arg, where X is any amino acid; the protease cleaves immediately C-terminal to the canonical sequence. For example, in some cases, an amino acid sequence comprising an S1 ligand-inducible proteolytic cleavage site can have the amino acid sequence GRRRRELDPM (SEQ ID NO:1), where cleavage occurs between the “RE” sequence. As another example, an amino acid sequence comprising an S1 ligand-inducible proteolytic cleavage site can have the amino acid sequence RQRRELDPM (SEQ ID NO:2), where cleavage occurs between the “RE” sequence.

In some cases, the fusion protein polypeptide includes an S2 ligand-inducible proteolytic cleavage site. An S2 ligand-inducible proteolytic cleavage site can be located within the HD-C segment. In some cases, the S2 ligand-inducible proteolytic cleavage site is an ADAM-17-type protease cleavage site. An ADAM-17-type protease cleavage site can comprise an Ala-Val dipeptide sequence, where the enzyme cleaves between the Ala and the Val. For example, in some cases, amino acid sequence comprising an S2 ligand-inducible proteolytic cleavage site can have the amino acid sequence KIEAVKSE (SEQ ID NO:3), where cleavage occurs between the “AV” sequence. As another example, an amino acid sequence comprising an S2 ligand-inducible proteolytic cleavage site can have the amino acid sequence KIEAVQSE (SEQ ID NO: 4), where cleavage occurs between the “AV” sequence.

In some cases, the fusion protein includes an S3 ligand-inducible proteolytic cleavage site. An S3 ligand-inducible proteolytic cleavage site can be located within the TM domain. In some cases, the S3 ligand-inducible proteolytic cleavage site is a gamma-secretase (γ-secretase) cleavage site. A γ-secretase cleavage site can comprise a Gly-Val dipeptide sequence, where the enzyme cleaves between the Gly and the Val. For example, in some cases, an S3 ligand-inducible proteolytic cleavage site has the amino acid sequence VGCGVLLS (SEQ ID NO:5), where cleavage occurs between the “GV” sequence. In some cases, an S3 ligand-inducible proteolytic cleavage site comprises the amino acid sequence GCGVLLS (SEQ ID NO:6).

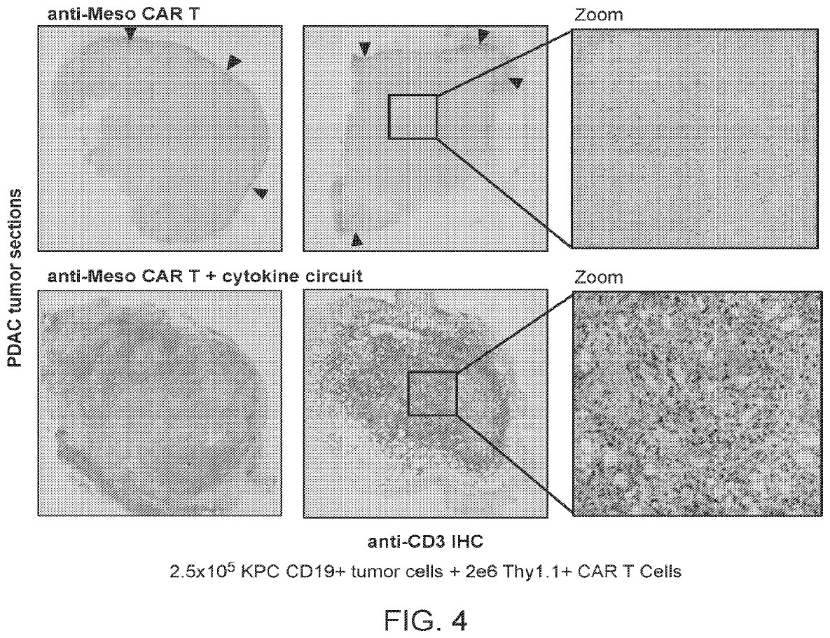

In some cases, the fusion protein polypeptide lacks an S1 ligand-inducible proteolytic cleavage site. In some cases, the Notch receptor polypeptide lacks an S2 ligand-inducible proteolytic cleavage site. In some cases, the Notch receptor polypeptide lacks an S3 ligand-inducible proteolytic cleavage site. In some cases, the Notch receptor polypeptide lacks both an S1 ligand-inducible proteolytic cleavage site and an S2 ligand-inducible proteolytic cleavage site. In some cases, the Notch receptor polypeptide includes an S3 ligand-inducible proteolytic cleavage site; and lacks both an S1 ligand-inducible proteolytic cleavage site and an S2 ligand-inducible proteolytic cleavage site. Examples are depicted schematically in FIG. 4G.

In other embodiments, the fusion protein may have an vWF A2 sequence or a variation thereof, an ADAMTS13 cleavage site (which may be described by the consensus sequence HEXXHXXGXXHD (SEQ ID NO:7); Crawley, Blood. 2011 118:3212-21), and an S3 or γ-secretase cleavage site, although many other arrangements exist. In some embodiments, the switch may contain components that are borrowed from Notch. In other embodiments, the switch may not contain components that are from Notch.

In some embodiments, the transmembrane domain of the fusion protein may contain a γ-secretase cleavage site comprising a Gly-Val dipeptide sequence, since Zhu et al (2021 bioRxiv) has shown that the SNIPRs (which are a type of BTTS) that have a transmembrane domain that contains a γ-secretase cleavage site do not require an ADAM cleavage site.

For simplicity, BTTSs, including but not limited to chimeric notch receptor polypeptides, are described primarily as single polypeptide chains. However, BTTSs, including chimeric notch receptor polypeptides, may be divided or split across two or more separate polypeptide chains where the joining of the two or more polypeptide chains to form a functional BTTS, e.g., a chimeric notch receptor polypeptide, may be constitutive or conditionally controlled. For example, constitutive joining of two portions of a split BTTS may be achieved by inserting a constitutive heterodimerization domain between the first and second portions of the split polypeptide such that upon heterodimerization the split portions are functionally joined.

Useful BTTSs that may be employed in the subject methods include, but are not limited to modular extracellular sensor architecture (MESA) polypeptides. A MESA polypeptide comprises: a) a ligand binding domain; b) a transmembrane domain; c) a protease cleavage site; and d) a functional domain. The functional domain can be a transcription regulator (e.g., a transcription activator, a transcription repressor). In some cases, a MESA receptor comprises two polypeptide chains. In some cases, a MESA receptor comprises a single polypeptide chain. Non-limiting examples of MESA polypeptides are described in, e.g., U.S. Patent Publication No. 2014/0234851; the disclosure of which is incorporated herein by reference in its entirety.

Useful BTTSs that may be employed in the subject methods include, but are not limited to polypeptides employed in the TANGO assay. The subject TANGO assay employs a TANGO polypeptide that is a heterodimer in which a first polypeptide comprises a tobacco etch virus (Tev) protease and a second polypeptide comprises a Tev proteolytic cleavage site (PCS) fused to a transcription factor. When the two polypeptides are in proximity to one another, which proximity is mediated by a native protein-protein interaction, Tev cleaves the PCS to release the transcription factor. Non-limiting examples of TANGO polypeptides are described in, e.g., Barnea et al. (Proc Natl Acad Sci USA. 2008 Jan. 8; 105 (1): 64-9); the disclosure of which is incorporated herein by reference in its entirety.

Useful BTTSs that may be employed in the subject methods include, but are not limited to von Willebrand Factor (vWF) cleavage domain-based BTTSs, such as but not limited to e.g., those containing a unmodified or modified vWF A2 domain. A subject vWF cleavage domain-based BTTS will generally include: an extracellular domain comprising a first member of a binding pair; a von Willebrand Factor (vWF) cleavage domain comprising a proteolytic cleavage site; a cleavable transmembrane domain and an intracellular domain. Non-limiting examples of vWF cleavage domains and vWF cleavage domain-based BTTSs are described in Langridge & Struhl (Cell (2017) 171 (6): 1383-1396); the disclosure of which is incorporated herein by reference in its entirety.

Useful BTTSs that may be employed in the subject methods include, but are not limited to chimeric Notch receptor polypeptides, such as but not limited to e.g., synNotch polypeptides, non-limiting examples of which are described in PCT Pub. No. WO 2016/138034, U.S. Pat. Nos. 9,670,281, 9,834,608, Roybal et al. Cell (2016) 167 (2): 419-432, Roybal et al. Cell (2016) 164 (4): 770-9, and Morsut et al. Cell (2016) 164 (4): 780-91; the disclosures of which are incorporated herein by reference in their entirety.

Anti-FAP antibodies that could be employed in the present fusion protein are numerous and include those described by Mersmann et al (Int J Cancer 2001 92:240-8), Zhang et al (FASEB J. 2013 27:581-589), Brocks et al (Molecular Medicine 2001 7:461-469), Schmidt et al (European Journal of Biochemistry 2001 268:1730-8) WO2016110598, WO2016116399, WO2014055442, US20090304718 and U.S. Pat. No. 10,253,110, which are incorporated by reference for a description of at least the CDRs of those antibodies.

The binding domain of the BTTS may be specific for any of the antigens listed in Table 1, for example. In some embodiments, a binding domain of the BTTS may have HC and LC CDR1, 2 and 3 sequences that are identical to or similar (i.e., may contain up to 5 amino acid substitutions, e.g., up to 1, up to 2, up to 3, up to 4 or up to 5 amino acid substitutions, collectively) to the CDRs of any of the antibodies listed in the publication cited in the table below, which publications are incorporated by reference for those sequences. The framework sequence could be humanized, for example. In some embodiments, the binding domain of the BTTS may have HC and LC variable regions that are at least 90%, at least 95%, at least 98% or at least 99% identical to a pair of HC and LC sequences listed in the publication cited in the table below, which publications are incorporated by reference for those sequences.

| Antigen binding domain | Exemplary sources of antigen binding sequences |
|---|---|
| Mesothelin (MSLN) | US 2021/0290676, US 2021/0284728 A1, US 2021/0275584 |
| | A1, Feng et al., Mol. Cancer Ther. 8(5): 1113-1118 (2009), US |
| | 2021/0269537 A1, US 2021/0252122 A1, US 2021/0230242 |
| | A1, US 2021/0155702 A1, US 2021/0137977 A1, US |
| | 2021/01016620 A1 |
| FAP | US 2021/0252122 A1, Kakarla et al. Mol Ther. 2013 |
| | August; 21(8): 1611-20, Wang et al. Cancer Immunol Res. 2015 |
| | July; 3(7): 815-826, Petrausch et al. BMC Cancer. 2012; 12: |
| | 615, Tran et al. J Exp Med. 2013 Jun. 3; 210(6): 1125-35. |
| Her2 | US 2021/0299269, US 2021/0290676, US 2021/0137977 A1, |
| | US 2021/01016620 A1, US 2021/0299172 A1 |
| Trop2 | US 2021/0290676, Zhao et al. Am J Cancer Res. 2019; 9(8): |
| | 1846-1856., Bedoya et al. Cytotherapy 2019 May; 21(5): S11- |
| | 12., Sayama et al. Mol Med Rep. 2021 February; 23(2): 92. |
| GPC3 | US 2021/0261646 A1, US 2021/0137977 A1, US |
| | 2021/01016620 A1, Li et al. Am J Transl Res. 2021 Jan. |
| | 15; 13(1): 156-167., Batra et al. Cancer Immunol Res. 2020 |
| | March; 8(3): 309-320. |
| MUC1 | US 2021/0269547 A1, US 2021/0155702 A1, Supimon et al. |
| | Sci Rep. 2021 Mar. 18; 11(1): 6276., Zhou et al. Front Immunol. |
| | 2019 May 24; 10: 1149., Mei et al. Cancer Med. 2020 |
| | January; 9(2): 640-652. |
| ROR1 | US 2021/0290676, Wallstabe et al JCI Insight. 2019 Sep. 19; |
| | 4(18): e126345, US 2021/0137977 A1, Prussak et al. J. Clin. |
| | Oncol. 2020; 38, no. 6_suppl, Srivastava et al. Cancer Cell. |
| | 2021 Feb. 8; 39(2): 193-208. e10. |
| EPCAM | US 2021/0290676, US 2021/0284728 A1, US 2021/0269547 |
| | A1, Qin et al. Oncoimmunology. 2020 Aug. 15; 9(1): 1806009., |
| | Deng et al. BMC Immunol. 2015 Jan. 31; 16(1): 1. |
| ALPPL2 | Su et al Cancer Res. 2020 Oct. 15; 80(20): 4552-4564., |
| | Hyrenius-Wittsten et al. Sci Transl Med. 2021 Apr. |
| | 28; 13(591): eabd8836., WO2017095823A1 |
| PSMA | US 2021/0290676, US 2021/0284728 A1, US 2021/0269547 |
| | A1, US 2021/0252122 A1, US 2021/0137977 A1, US |
| | 2021/0113615 A1 |
| PSCA | US 2021/0290676, US 2021/0269547 A1, Wu et al. Biomark |
| | Res. 2020 Jan. 28; 8: 3., Dorff et al. J. Clin. Oncol. 2020; 38, no. |
| | 6_suppl, US 2020/0308300 |
| EGFRviii | US 2021/0290676, US 2021/0252122 A1, US 2021/0137977 |
| | A1, O'Rourke et al. Sci Transl Med. (2017) 9: eaaa0984, Abbott |
| | et al. Clin Transl Immunology. 2021 May 9; 10(5): e1283. |
| EGFR | US 2021/0290676, US 2021/0269547 A1, US 2021/0155702 |
| | A1, Xia et al. Clin Transl Immunology. 2020 May |
| | 3; 9(5): e01135., Li et al. Cell Death Dis. 2018 February; 9(2): 177., |
| | Liu et al. Clinical Trial Cytotherapy. 2020 October; 22(10): 573-580. |
| Claudin 18.2 | US 2021/0252122 A1, Jiang et al. J Natl Cancer Inst. 2019 Apr. |
| | 1; 111(4): 409-418., Chin et al. J Cancer Res. 2020 April; 32(2): |
| | 263-270., Zhan et al. J. Clin. Oncol. 2019, 37, 2509., Singh et |
| | al. J Hematol Oncol. 2017; 10: 105. |
| GD2 | US 2021/0290676, Seitz et al. Oncoimmunology. 2020; 9(1): |
| | 1683345., Chulanetra et al. Am J Cancer Res. 2020; 10(2): |
| | 674-687., Sujjitjoon et al. Transl Oncol. 2021 |
| | February; 14(2): 100971, Andersch et al. BMC Cancer. 2019 Sep. |
| | 9; 19(1): 895. |

Alternatively, a tissue-specific antigen can be used in some embodiments, many of which are known. For example, I

New antigen binding domains may also be generated in the form of immunoglobulin single variable (ISV) domains. The ISV domains may be generated using any suitable method. Suitable methods for the generation and screening of ISVs include without limitation, immunization of dromedaries, immunization of camels, immunization of alpacas, immunization of sharks, yeast surface display, etc. Yeast surface display has been successfully used to generate specific ISVs as shown in McMahon et al. (2018) Nature Structural Molecular Biology 25 (3): 289-296 which is specifically incorporated herein by reference.

Immunoglobulin sequences, such as antibodies and antigen binding fragments derived there from (e.g., immunoglobulin single variable domains or ISVs) are used to specifically target the respective antigens disclosed herein. The generation of immunoglobulin single variable domains such as e.g., VHHs or ISV may involve selection from phage display or yeast display, for example ISV can be selected by utilizing surface display platforms where the cell or phage surface display a synthetic library of ISV, in the presence of tagged antigen. A fluorescent secondary antibody directed to the tagged antigen is added to the solution thereby labeling cells bound to antigen. Cells are then sorted using any cell sorting platform of interest e.g., magnetic-activated cell sorting (MACS) or fluorescence-activated cell sorting (FACS). Sorted clones are amplified, resulting in an enriched library of clones expressing ISV that bind antigen. The enriched library is then re-screened with antigen to further enrich for surface displayed antigen binding ISV. These clones can then be sequenced to identify the sequences of the ISV of interest and further transferred to other heterologous systems for large scale protein production.

Expression of the BTTS in the cell may be constitutive or inducible, e.g., by binding of another BTTS to an antigen on another cell in the tumor.

Examples of transcriptional activators that can be part of the fusion protein are numerous and include artificial transcription factors (ATFs) such as, e.g., Zinc-finger-based artificial transcription factors (including e.g., those described in Sera T. Adv Drug Deliv Rev. 2009 61 (7-8): 513-26; Collins et al. Curr Opin Biotechnol. 2003 14 (4): 371-8; Onori et al. BMC Mol Biol. 2013 14:3. In some cases, the transcriptional activator may contain a GAL4 DNA binding domain, which binds to the Gal4 responsive UAS, which has been well characterized in the art. Examples of suitable transcriptional activators include GAL4-VP16 and GAL4-VP64, although many others could be used. As would be appreciated, the identity of the transcription activators may vary. In some embodiments, the transcription factor may have a DNA binding domain that binds to a corresponding promoter sequence and an activation domain. In many embodiments, the DNA binding domain of the first and second transcription factors may be independently selected from Gal4-, LexA-, Tet-, Lac-, dCas9-, zinc-finger- and TALE-based transcription factors. TALE- and CRISPR/dCas9-based transcription factors are described in Lebar (Methods Mol Biol. 2018 1772:191-203), among others. The binding sites for such domains are well known or can be designed at will. The first and second transcription factors can have any suitable activation domain, e.g., VP16, VP64, Ela, Sp1, VP16, CTF, GAL4 among many others.

### Pro-Inflammatory Proteins

As noted above, binding of BTTS to an antigen on the surface of another cell activates expression of the pro-inflammatory protein. In these embodiments, binding of the binding domain of the BTTS to the tissue- or cancer-associated marker on another cell in the tumor induces proteolytic cleavage of the one or more force-dependent cleavage sites to release the transcriptional activator. The released transcriptional activator then binds to a promoter that drives the expression of the pro-inflammatory protein, thereby inducing expression of the pro-inflammatory protein. The general principles of a circuit are described in WO 2016/138034, U.S. Pat. Nos. 9,670,281, 9,834,608, Roybal et al. Cell (2016) 167 (2): 419-432, Roybal et al. Cell (2016) 164 (4): 770-9, and Morsut et al. Cell (2016) 164 (4): 780-91, among others.

The pro-inflammatory protein will be secreted from the cell. In these embodiments, the circuit may comprise a nucleic acid containing a promoter that is activated by the released transcriptional activator, and a coding sequence encoding a pro-inflammatory protein. In this disclosure, the term “pro-inflammatory protein” is intended to encompass any cytokine that have a pro-inflammatory activity (e.g., IL-2, CCL-21, IL-12, IL-7, IL-15 and IL-21, etc.), as well as non-natural or “engineered” cytokines that have pro-inflammatory activity such as super IL-2 (see, e.g., Levin et al Nature 2012 484:529-533, which has the following amino acid substitutions L80F, R81D, L85V, 186V, and 192F relative to wild type), mini-TGF-Beta (which blocks TGF-Beta signaling) and DR-18 (an IL-18 variant), etc.). Engineered cytokines include superkines, which often have up to 10 amino acid substitutes relative to a natural cytokine, as well as natural cytokines that have been truncated, and dominant variants. Cytokines of interest include selected from IL-2, IL-12, IL-15, IL-7, CD40L, or a non-natural variant of IL-2, IL-12, IL-15, IL-7, CD40L that has pro-inflammatory activity. Cytokines include “ortho” cytokines that can be paired with a receptor in the immune cell (see, e.g., Sockolosky et al. 2018). Other pro-inflammatory proteins include immune checkpoint inhibitors, including molecules that block interactions with PD1, CTLA4, BTLA, CD160, KRLG-1, 2B4, Lag-3, Tim-3 and other immune checkpoints. See, e.g., Odorizzi and Wherry (2012) J. Immunol. 188:2957; and Baitsch et al. (2012) PLOSOne 7: e30852. For example, an anti-PD1 antibody, an anti-PDL1 antibody a decoy resistant IL-18 and a dominant negative TGF-β, a dominant negative TGFβ receptor, or a TGFβ inhibitor/agonist could be used. In some embodiments, activation of the circuit may induce the express of a combination of pro-inflammatory proteins.

Sources for exemplary pro-inflammatory proteins are listed below.

| Payload | Exemplary sources of payload sequences |
|---|---|
| IL2 | NCBI Gene ID: 3558 |
| super-2 | Levin et al. Nature. 2012 Apr. 26; 484(7395): 529-533. |
| ortho2 | Sockolosky et al. Science. 2018 Mar. 2; 359(6379): 1037-1042. |
| IL15 | NCBI Gene ID: 3600 |
| Anti-PD1 | Pembrolizumab, Nivolumab, Cemiplimab, Dostarlimab, JTX- |
| | 4014, Spartalizumab, Camrelizumab, Sintilimab, Tislelizumab, |
| | Toripalimab, INCMGA00012, AMP-224, AMP-514 |
| Anti-PDL1 | Atezolizumab, Avelumab, Durvalumab, KN035, CK-301, |
| | AUNP12, CA-170, BMS-986189 |
| IL7 | NCBI Gene ID: 3574 |
| CD40L | NCBI Gene ID: 959 |
| Decoy Resistant IL-18 | Zhou et al. Nature. 2020 July; 583(7817): 609-614. |
| (DRIL-18) | |
| Dominant negative TGFb | Kloss et al. Mol Ther. 2018 Jul. 5; 26(7): 1855-1866., Wieser et |
| | al. Mol Cell Biol. 1993 December; 13(12): 7239-47. |

As would be appreciated, pro-inflammatory proteins are secreted from the cell and their coding sequence will encode a secretion signal.

As noted above, in some embodiments the immune cell may additionally express a recombinant receptor for the pro-inflammatory protein, which further enhances the immune cell's response. For example, if the pro-inflammatory protein is an “ortho2”, then the immune cell may additionally express a receptor for that pro-inflammatory protein.

### Cytotoxic Immune Receptors

As noted above, the immune receptor may be a chimeric antigen receptor (CAR) or an engineered T cell receptor (TCR). In any embodiment, the immune receptor may be constitutively expressed. In other embodiments, expression of the immune receptor may be under the control of the BTTS. In either event, the immune receptor will be expressed on the surface of the cell and will be activated by binding to an antigen that is expressed by the cancerous cells, e.g., by the malignant cells, e.g., any of the antigens listed in Table 1, for example. Binding domains for many of these antigens are described above.

Binding of the immune receptor to its cognate antigen activates the immune cell. CARs can be designed in several ways (see, generally, e.g., Guedan et al, Methods and Clinical Development 2019 12:145-156) and may include an extracellular domain that contains an antigen binding domain such as a scFv or nanobody, a hinge, a transmembrane region (which may be derived from CD4, CD8a, or CD28), a costimulatory signaling domains (which may be derived from the intracellular domains of the CD28 family (e.g., CD28 and ICOS) or the tumor necrosis factor receptor (TNFR) family of genes (e.g., 4-1BB, OX40, or CD27), and an ITAM domain, e.g., the signaling domain from the zeta chain of the human CD3 complex (CD3zeta). In practice, any of these domains may be a variation of a wild type sequence. In practice, any of these sequences may be a variant of a wild type sequence, e.g., a sequence that is at least 90%, 95, or 98% identical a sequence described in WO2014127261, for example. Sources for exemplary sequences that can bind to Mesothelin, FAP, Her2, Trop2, GPC3, MUC1, ROR1, EPCAM, ALPPL2, PSMA, PSCA, EGFRviii, EGFR, Claudin18.2, and GD2 are listed above. However, sequences that bind to other antigens are known and/or can be readily made.

As noted above, the immune receptor may be constitutively expressed (in which case its coding sequence will be operably linked to a constitutive promoter, i.e., a promoter that is always “on” in the cell), or induced by activation of the BTTS. In the latter case, the coding sequence for the pro-inflammatory protein and the coding sequence for the immune receptor may be both operably linked a single promoter (in which case the coding sequences may be separated by an IRES sequence, although other systems such as bidirectional promoters can be used), or they may be linked to different promoters, which may or may not have different sequences.

In alternative embodiments, the BTTS and immune receptor do not need to be in the same immune cell. In some embodiments, the BTTS is expressed one cell and immune receptor is expressed on the other. These cells may be administered together.

In any embodiment, the immune receptor and/or the BTTS may be a hybrid molecule and, in some cases, may be a hybrid between immune receptor and a BTTS.

### Methods of Treatment

A method of treatment for a cancer associated with a solid tumor is described below. In general terms, this method may comprise administering a cell described above to a subject that has a solid tumor. In some embodiments, primary immune cells (e.g., T cells or NK cells, etc.) may be purified from an individual, constructs encoding the above proteins may be introduced into the cells ex vivo, and the recombinant cells may be expanded and administered to the subject, e.g., by injection. Alternatively, allogeneic immune cells may be used.

The antigens to which the immune receptor and BTTS bind depend on which cancer is being treated. The following table provides a list of 18 cancers that are associated with solid tumors, and the antigens that are frequently expressed by those tumors. Selection of the binding sequences for the immune receptor and BTTS may be based on Table 2 below. These methods may be used to treat metastasized cancers, too, e.g., any of the cancers listed below, which has metastasized to another tissue.

TABLE 2

| Cancer | Antigens |
|---|---|
| Lung Cancer | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, Mesothelin, FAP, HER2, |
| | EphA2, PMSA, CD70, GD2, EGFR, EGFRviii, PSCA, MET, AXL, |
| | MCAM, Trop2, MUC1, ROR1, Claudin18.2, GPC3, CA9, EPCAM, |
| | FOLR1, L1CAM, CD133, CEACAM5, MUC16, PD-L1, ROR2 |
| Colorectal Cancer | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, FAP, EphA2, PMSA, |
| | CD70, ALPPL2, HER2, EGFR, PSCA, MET, AXL, MCAM, Trop2, |
| | MUC1, ROR1, Claudin18.2, GPC3, CA9, EPCAM, FOLR1, L1CAM, |
| | CD133, CEACAM5, PD-L1, ROR2 |
| Pancreatic Cancer | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1Mesothelin, FAP, IL13Ra2, |
| | PMSA, ALPPL2, HER2, EphA2, EGFR, PSCA, MET, AXL, MCAM, |
| | Trop2, MUC1, ROR1, Claudin18.2, CA9, EPCAM, FOLR1, L1CAM, |
| | CD133, CEACAM5, MUC16, PD-L1, ROR2 |
| Prostate Cancer | ALK, VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, FAP, EphA2, PMSA, |
| | HER2, EGFR, PSCA, AXL, MCAM, Trop2, MUC1, ROR1, EPCAM, |
| | CD133, PD-L1, ROR2 |
| Liver and biliary | VEGFR2, DLL3, DLL4, NECTIN1, NECTIN2, NECTIN3, |
| tract cancers | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1Mesothelin, FAP, EGFR, |
| | EGFRviii, EphA2, MET, AXL, MCAM, MUC1, ROR1, Claudin18.2, |
| | GPC3, CA9, EPCAM, CD133, CEACAM5, PD-L1, ROR2 |
| Bladder Cancer | ALK, VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, FAP, PMSA, , HER2, |
| | EGFR, EphA2, PSCA, MET, AXL, MCAM, Trop2, MUC1, ROR1, |
| | GPC3, CA9, EPCAM, L1CAM, CD133, CEACAM5, PD-L1, ROR2 |
| Brain Cancer | ALK, VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPMIBCAN, MOG, CSPG5, |
| | PTPRZ1, EGFRviii, IL13Ra2, EphA2, CD70, GD2, EGFR, HER2, |
| | MET, AXL, MCAM, ROR1, CA9, L1CAM, PD-L1, ROR2 |
| Esophageal Cancer | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, FAP, EphA2, AXL, |
| | MCAM, PD-L1, ROR2 |
| Ovarian Cancer | ALK, VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, Mesothelin, FAP, HER2, |
| | Il13Ra2, EphA2, ALPPL2, GD2, EGFR, AXL, MCAM, Trop2, |
| | ROR1, GPC3, CA9, EPCAM, FOLR1, DLL3, DLL4, NECTIN1, |
| | NECTIN2, NECTIN3, NECTIN4, L1CAM, CD133, CEACAM5, |
| | MUC16, PD-L1, ROR2 |
| Kidney Cancer | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, FAP, EphA2, PMSA, |
| | CD70, GD2, HER2, EGFR, PSCA, MET, AXL, MCAM, Trop2, |
| | MUC1, ROR1, Claudin18.2, GPC3, CA9, EPCAM, FOLR1, L1CAM, |
| | CD133, PD-L1, ROR2 |
| Melanoma | MLANA (MART1), TYRP-1, ALK, VEGFR2, DLL3, DLL4, |
| | NECTIN1, NECTIN2, NECTIN3, NECTIN4, CD44v6, NKG2D, |
| | MAGE family (A-L types), WT1, KREMEN2, GRIN2D, CELSR3, |
| | KISS1R, CDH6, TREM1, MC1R, LIFR, PRR7, PTPRN, CAGNG7, |
| | TRPM1, FAP, lL13RA2, EphA2, CD70, MET, AXL, MCAM, Trop2, |
| | ROR1, L1CAM, PD-L1, ROR2 |
| Gastric/Stomach | VEGFR2, DLL3, DLL4, NECTIN1, NECTIN2, NECTIN3, |
| Cancer | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, FAP, HER2, CD70, |
| | ALPPL2, EphA2, EGFR, PSCA, MET, AXL, MCAM, Trop2, MUC1, |
| | ROR1, Claudin18.2, GPC3, CA9, EPCAM, FOLR1, L1CAM, CD133, |
| | CEACAM5, MUC16, PD-L1, ROR2 |
| Breast Cancer | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1, FAP, HER2, EphA2, |
| | PMSA, CD70, GD2, EGFR, PSCA, MET, AXL, MCAM, Trop2, |
| | MUC1, ROR1, Claudin18.2, GPC3, CA9, EPCAM, FOLR1, L1CAM, |
| | CD133, CEACAM5, PD-L1, ROR2 |
| Mesothelioma | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1Mesothelin, ALPPL2, AXL, |
| | Claudin18.2, PD-L1, ROR2 |
| Uterine | VEGFR2, DLL3, DLL4, NECTIN1, NECTIN2, NECTIN3, |
| | NECTIN4, CD44v6, NKG2D, MAGE family (A-L types), WT1, |
| | KREMEN2, GRIN2D, CELSR3, KISS1R, CDH6, TREM1, MC1R, |
| | LIFR, PRR7, PTPRN, CAGNG7, TRPM1HER2, EphA2, Trop2, |
| | MUC1, ROR1, GPC3, CA9, EPCAM, FOLR1, L1CAM, CD133, |
| | CEACAM5, MUC16, PD-L1, ROR2 |
| testicular | VEGFR2, DLL3, DLL4, NECTIN1, NECTIN2, NECTIN3, |
| | NECTIN4, EphA2, PMSA, ALPPL2, AXL, MCAM, ROR1, GPC3, |
| | EPCAM, L1CAM, CD133, PD-L1, ROR2 |
| head and neck | VEGFR2, DLL3, DLL4, NECTIN1, NECTIN2, NECTIN3, |
| (including thyroid) | NECTIN4, EphA2, FAP, HER2, EGFR, PSCA, MET, Trop2, MUC1, |
| | ROR1, GPC3, CA9, EPCAM, L1CAM, CD133, CEACAM5, MUC16, |
| | PD-L1, ROR2 |

Reference to “MAGE family” includes any of the MAGE family members listed in Table 2 of Weon et al (Curr Opin Cell Biol. 2015 37:1-8), particularly MAGE A1, MAGE A2, MAGE A3, MAGE A4, which are each associated with various solid tumors, e.g., NSCLC, melanoma, breast, ovarian and colon.

In some embodiments, an antigen may be selected from the following list: mesothelin, FAP, EGFRvIII, IL13RA2, EPHA2, PSMA (FOLH1), HER2, EGFR, PSCA, ALPPL2, GD2 (B4GALNT1), BCAN, MOG, CSPG5, CD70, MET, AXL, MCAM, DLL3, DLL4, nectin4, nectin2, nectin3, nectin1, and ALK.

In some embodiments, (a) the subject has a cancer selected from the cancers listed in Table 2; (b) the immune receptor is activated by binding to an antigen associated with that cancer in Table 2; and (c) the BTTS is independently activated by binding to either a tissue-specific antigen for the cancer or to an antigen associated with that cancer in Table 2. In these embodiments, binding of the immune receptor to its antigen activates the immune cell; a binding of the BTTS to its antigen activates expression of the pro-inflammatory protein and, optionally, the immune receptor if the immune receptor is not constitutively expressed in the cell.

In any embodiment, the antigen to which the immune receptor binds to and the antigen to which the BTTS binds to may be the same or different. As would be apparent, the immune receptor may be constitutively expressed or induced by activation of the BTTS.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

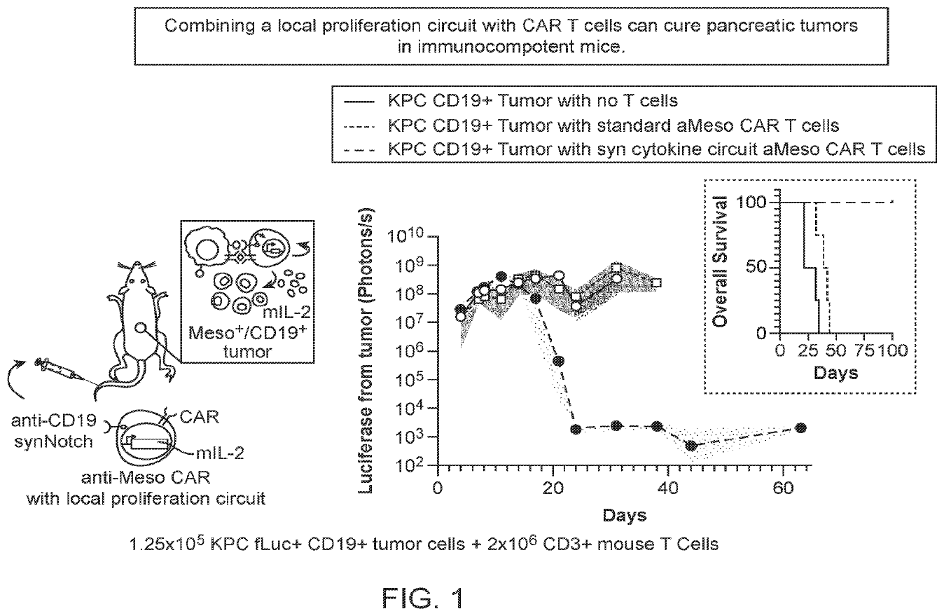

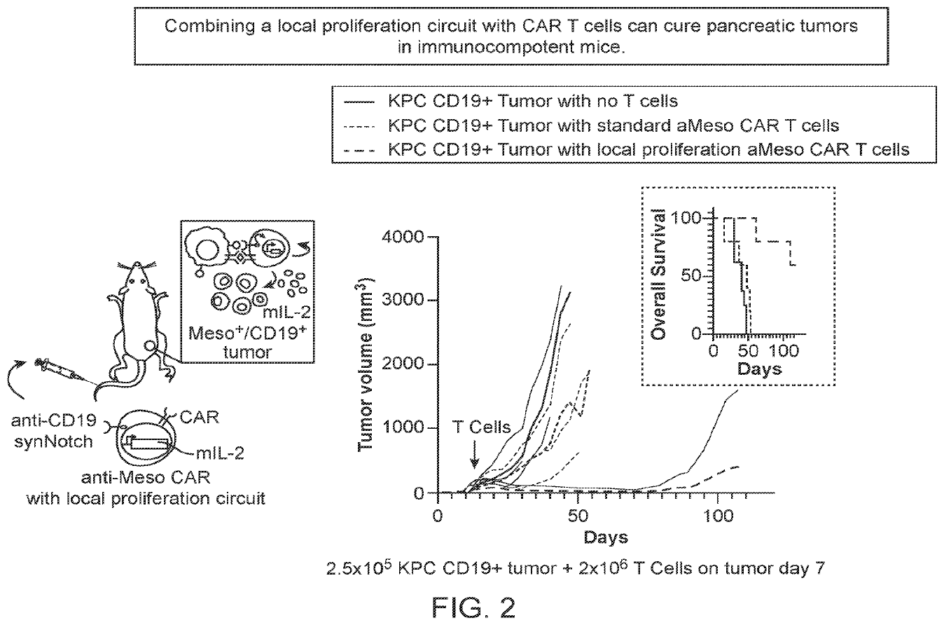

According to the data shown in FIGS. 1 and 2, there is both a significant improvement in tumor clearance as well as mouse survival using T cells that express an anti-Meso CAR and a synNotch-IL2 circuit compared to either no T cells or T cells that produce only the anti-Meso CAR. These experiments were done in vivo, in syngeneic pancreatic cancer models in immunocompetent mice.

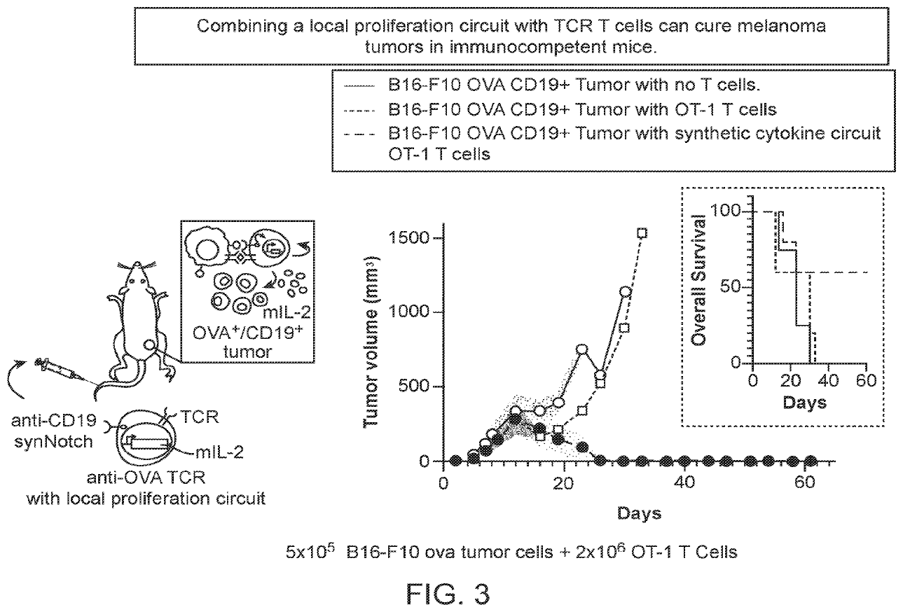

According to the data shown in FIG. 3, there is both a significant improvement in tumor clearance as well as mouse survival using T cells that express an anti-OVA TCR and a synNotch IL2 circuit compared to either no T cells or cells that produce only the anti-OVA CAR (no synNotch circuit). These experiments were done in vivo, in syngeneic melanoma cancer models in immunocompetent mice.

According to the data shown in FIG. 4, addition of a synNotch→IL2 circuit dramatically increases T cell infiltration into immune excluded tumor.

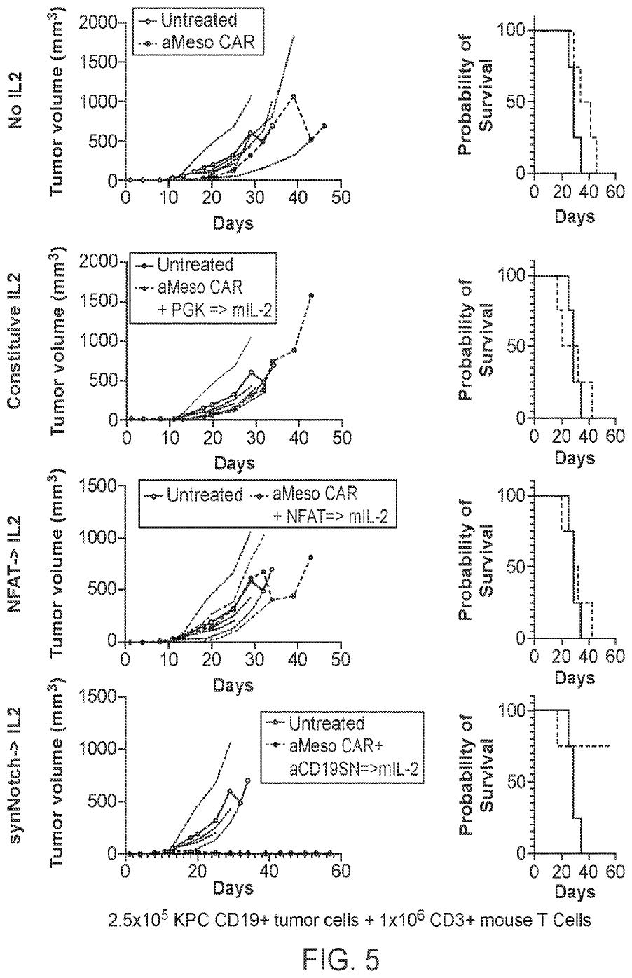

According to the data shown in FIG. 5, use of cells that have a synNotch→IL2 circuit provides better results that other ways to deliver IL2 in CAR T cells.

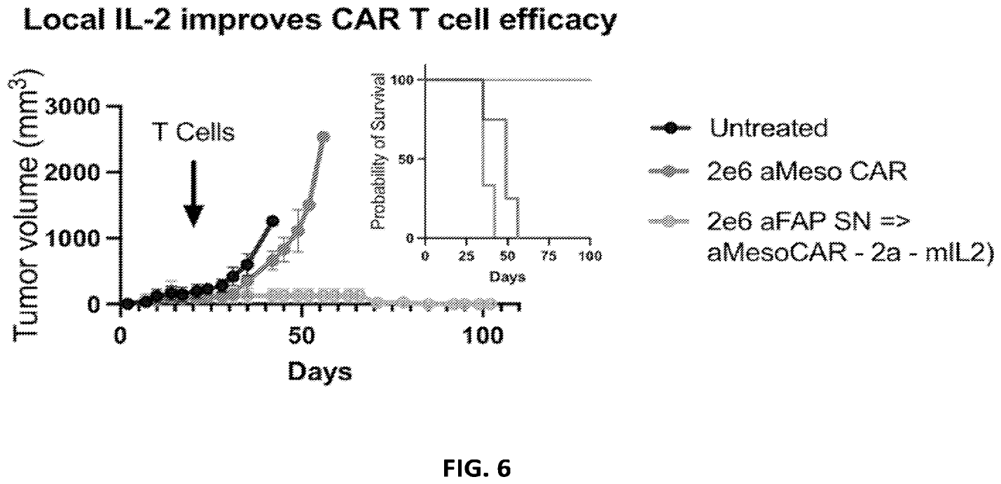

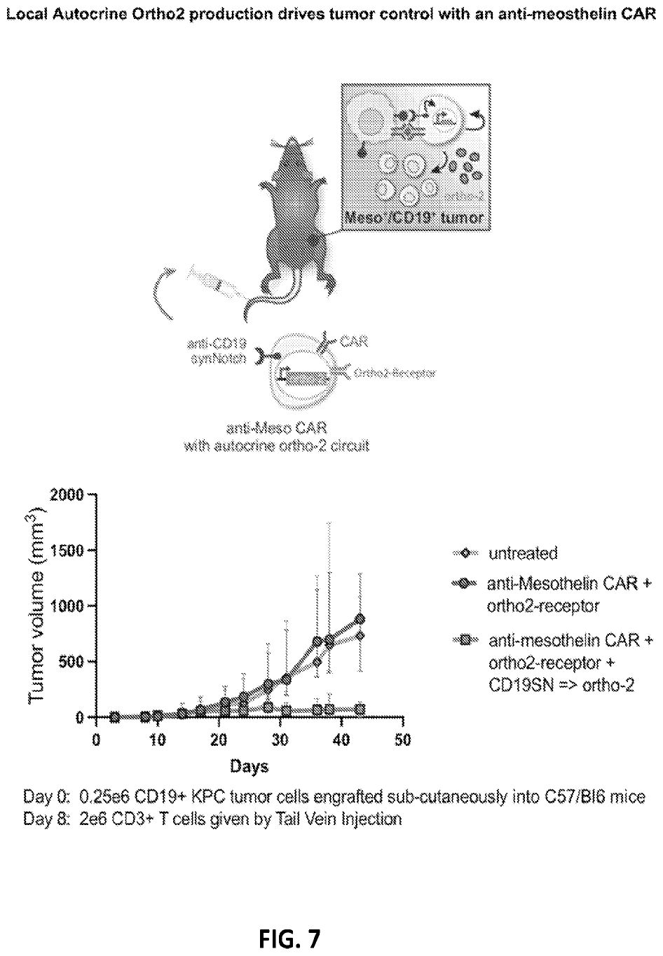

According to the data shown in FIG. 6, use of cells that have an anti-FAP synNotch→anti-MesoCAR+IL2 circuit is an effective way of delivering CAR and cytokine to a tumor.

According to the data shown in FIG. 6, improved control of syngeneic pancreatic cancer models in immunocompetent mice is improved using cells that produce an anti-Meso CAR and an ortho2 receptor chain as well as synNotch producing ortho IL-2. The advantage here is that ortho2 will not stimulate native T cells (or other immune cells), but will only stimulate the engineered T cells that express the ortho2 receptor.

In the following examples, engineered circuits in which tumor-specific synNotch receptors locally induce production of the inflammatory cytokine, interleukin-2 (IL-2). These cytokine delivery circuits can potently enhance CAR T cell infiltration and clearance of immune-excluded tumors (immunocompetent models of pancreatic cancer and melanoma) without systemic toxicity. The most effective IL-2 induction circuit acts in an autocrine and TCR/CAR-independent manner, bypassing suppression by host cells that either consume IL-2 or inhibit TCR signaling. These engineered autocrine cells are able to establish an effective foothold in the tumors, likely because synNotch-induced IL-2 production can cooperatively enable initiation of CAR-mediated T cell expansion and killing. Thus, it is possible to reconstitute synthetic T cell circuits that activate the outputs ultimately required for a robust anti-tumor response, but in a manner that evades key points of tumor suppression.

### Results

Engineering Synthetic IL-2 Circuits that Drive Local T Cell Proliferation Independent of T Cell Activation

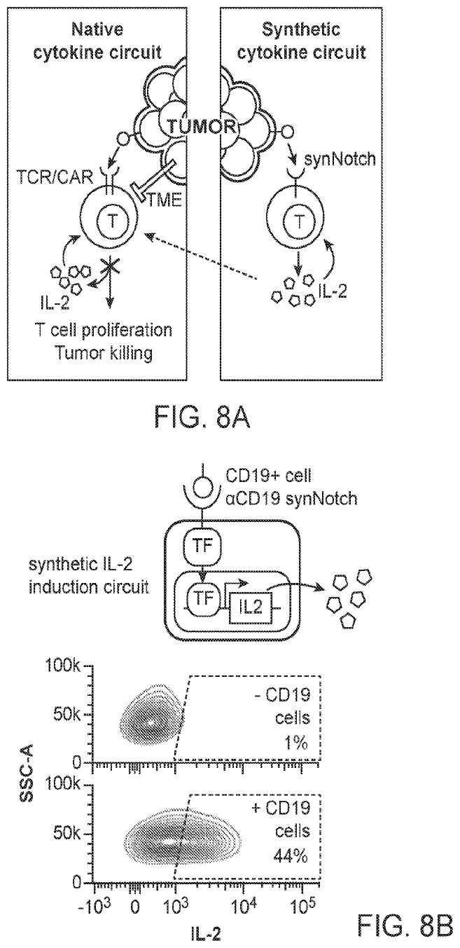

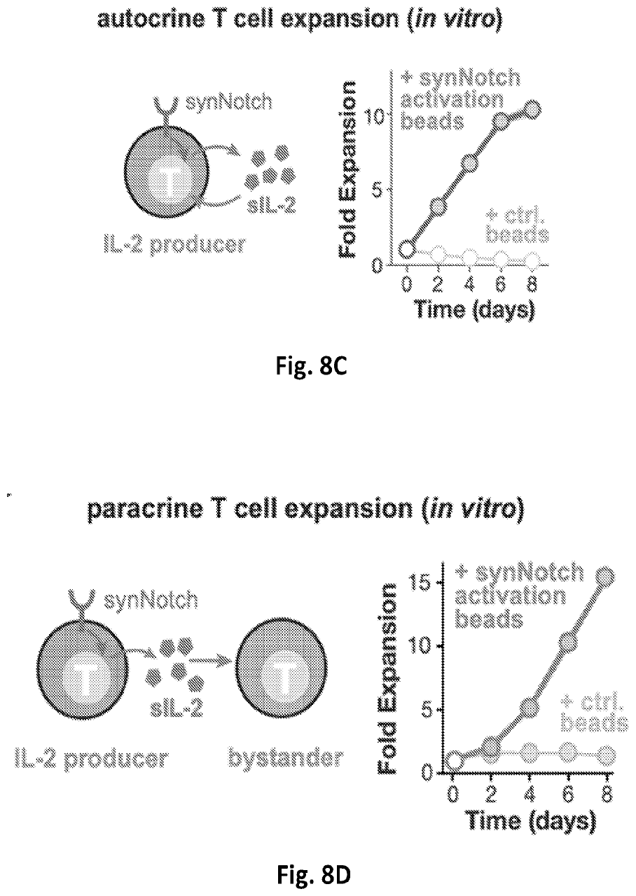

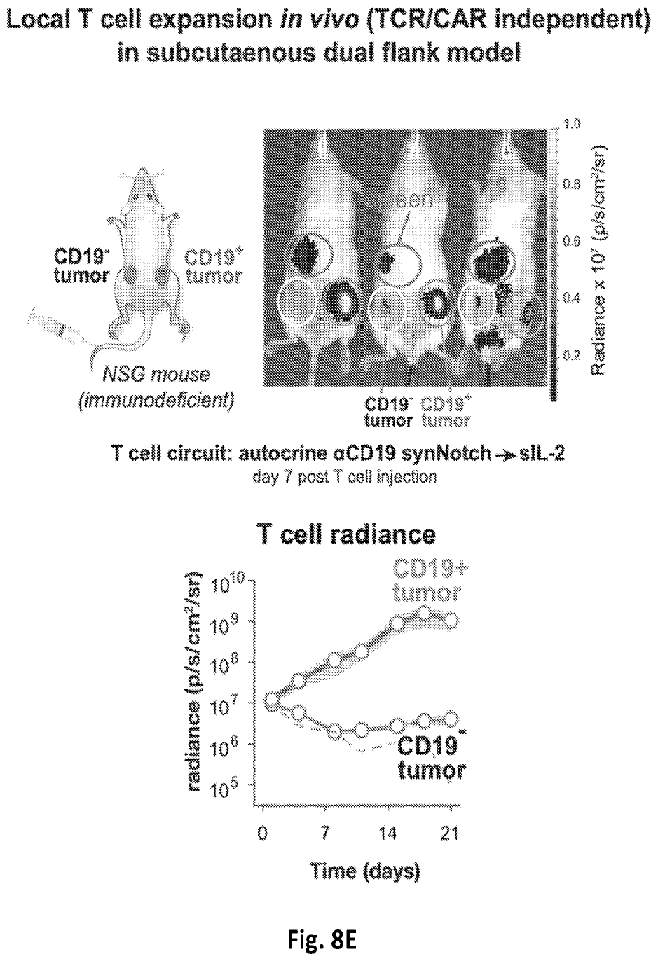

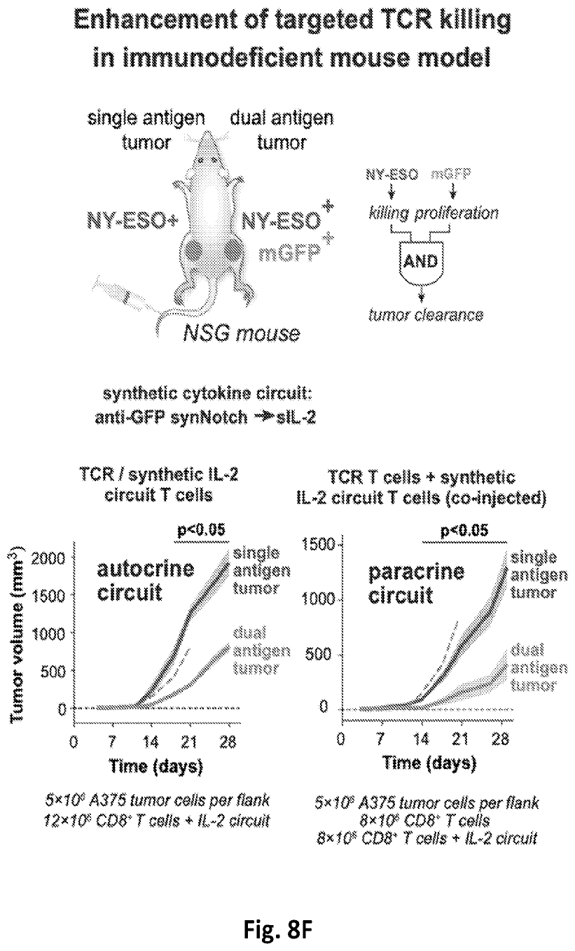

To design a tumor-induced synthetic IL-2 circuit in T cells, we used a synNotch sensor to induce the transcription of an IL-2 transgene (FIG. 8B). Briefly, synNotch receptors are chimeric receptors with a variable extracellular recognition domain, a Notch-based cleavable transmembrane domain, and an intracellular transcriptional domain (12, 13). Antigen binding induces intramembrane receptor cleavage, releasing the transcriptional domain to enter the nucleus and promote expression of a target transgene.

We built a prototype circuit in primary human T cells, using a synNotch receptor that recognizes the model antigen CD19, combined with a synNotch-responsive promoter driving expression of human IL-2 or an affinity-enhanced variant of IL-2 (known as super-2 or sIL-2) (21). As intended, stimulation of the synNotch receptor in vitro induced strong proliferation of the engineered cell population (FIG. 8C). Cells with the anti-CD19 synNotch→sIL-2 circuit could function in a paracrine manner, driving the proliferation of co-cultured non-engineered T cells (FIG. 8D) or NK cells (not shown) in vitro. The degree of proliferation was dependent on the type of gamma-chain cytokine payload, with significant T cell proliferation seen with production of either IL-2 or sIL-2 (not shown). Production of the homeostatic cytokine IL-7 (22) led to T cell survival with minimal expansion, while un-tethered IL-15 (23) had no effect. Thus, in vitro, a synNotch→sIL-2 circuit T cell can drive its own proliferation, as well as the proliferation of other co-cultured IL-2 responsive cells.

We then tested whether the synNotch→sIL-2 circuit could drive targeted expansion of human T cells in vivo, independent of CAR or TCR activation. We established a bilateral K562 tumor model in immunocompromised NOD scid gamma (NSG) mice, where only one flank tumor expressed the synNotch target antigen, CD19 (FIG. 8E). Human primary CD8+ T cells engineered with the anti-CD19 synNotch→sIL-2 circuit were tagged with enhanced firefly luciferase (eff-luc) and injected intravenously. Cells with the synthetic IL-2 circuit autonomously identified the target tumor (CD19⁺/right) and locally expanded approximately 100-fold within this tumor (FIG. 8E). In contrast, no off-target expansion was seen in the contralateral (CD19⁻) tumor. Flow cytometry analysis of tumor infiltrating lymphocytes (TILs) in the target and off-target tumor showed synNotch activation, T cell expansion, and proliferation only in the CD19⁺ tumor (not shown). The administered T cells have no CAR or TCR reactivity against tumors, thus the synthetic production of IL-2 alone did not result in killing of the K562 tumors in this immunodeficient NSG mouse model (not shown).

We also found that the anti-CD19 synNotch→sIL-2 circuit was also capable of driving T cell expansion in a paracrine (two-cell type) configuration, in this NSG mouse model. Here we co-injected a population of bystander T cells, which did not express the sIL-2 induction circuit but expressed luciferase to distinguish them from the synNotch→sIL-2 T cells. Co-injected into mice at a 1:1 ratio, the bystander cells also specifically expanded in the targeted (CD19⁺/right) tumor (not shown) where the synNotch receptor was locally activated (not shown). This paracrine T cell expansion was not observed in negative control experiments using synNotch T cells that either did not produce sIL-2 or did not recognize CD19 (not shown).

In summary, this work represents one of the first examples in which locally targeted T cell expansion can be induced in a manner uncoupled from TCR or CAR activation.

### Synthetic IL-2 Circuits can Enhance Targeted T Cell Cytotoxicity In Vivo

Many engineered T cell therapies show effective cytotoxicity in vitro but fail to show sufficient proliferation or persistence to achieve effective tumor control in vivo. For example, cells bearing the affinity-enhanced anti-NY-ESO-1 TCR are able to lyse A375 melanoma tumors in vitro (24), but have shown limited clinical benefit in patients or preclinical models (25). We hypothesized that the addition of a synthetic cytokine circuit producing IL-2 might enhance tumor control by NY-ESO-1 T cells. Moreover, these T cells might function as a new type of AND gate (26, 27), where a therapeutic T cell exhibits enhanced specificity by requiring two antigens to be present before triggering its full cytotoxic response (the TCR antigen required for T cell activation, and the synNotch antigen required for inducing IL-2 production). In this case, we used an anti-GFP synNotch→sIL-2 synthetic cytokine circuit. By requiring the presence of both the TCR antigen (NY-ESO-1) and the synNotch antigen (in this case, membrane-tethered GFP) (FIG. 8F), this cellular design strategy should further minimize off-target toxicity.

We examined the efficacy of anti-NY-ESO-1 TCR human T cells in NSG mice using a bilateral tumor model of a NY-ESO-1+melanoma (A375). Only one flank tumor was co-labelled with the synNotch-targeted model antigen (membrane-tethered GFP). Anti-NY-ESO-1 TCR-expressing T cells lacking the synthetic IL-2 circuit were largely ineffective at controlling the growth of both the single (NY-ESO+) and dual (NY-ESO+/GFP+) antigen tumors (not shown). However, when mice were treated with T cells simultaneously expressing both the anti-NY-ESO-1 TCR and the anti-GFP synNotch→sIL-2 circuit, the dual-targeted NY-ESO+/GFP+tumor now showed a significant reduction in tumor size (FIG. 8F). Similar tumor reduction was observed when IL-2 was provided in a paracrine configuration, by co-injection of one cell type only expressing the anti-NY-ESO-1 TCR and a second cell type only expressing the synthetic IL-2 circuit. Critically, in either the autocrine or paracrine configuration, the synthetic IL-2 circuit did not cause a reduction in the contralateral NY-ESO+/GFP tumor (lacking the synNotch ligand), highlighting the precisely targeted impact of the synthetic IL-2 circuit.

Using luciferase tracking of anti-NY-ESO-1 TCR T cells, we observed substantially increased intratumoral expansion of T cells only in tumors that were targeted by the synthetic IL-2 circuit (not shown). The synthetic IL-2 circuit was only activated in the targeted double antigen positive tumor (not shown), and we observed a significant increase in T cell activation markers in this targeted tumor (not shown). A synthetic IL-2 circuit T cell without co-delivery of a tumor reactive cytotoxic T cell population did not produce tumor control in these NSG mouse models (not shown).

### Autocrine Configuration of Synthetic IL-2 Circuit is Required in Immunocompetent Tumor Models

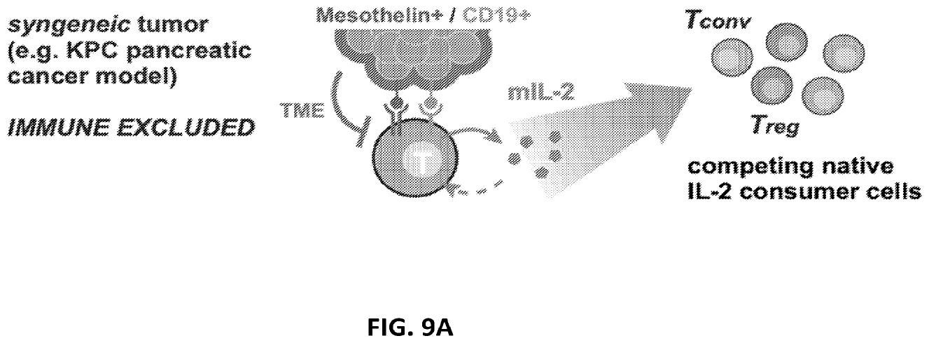

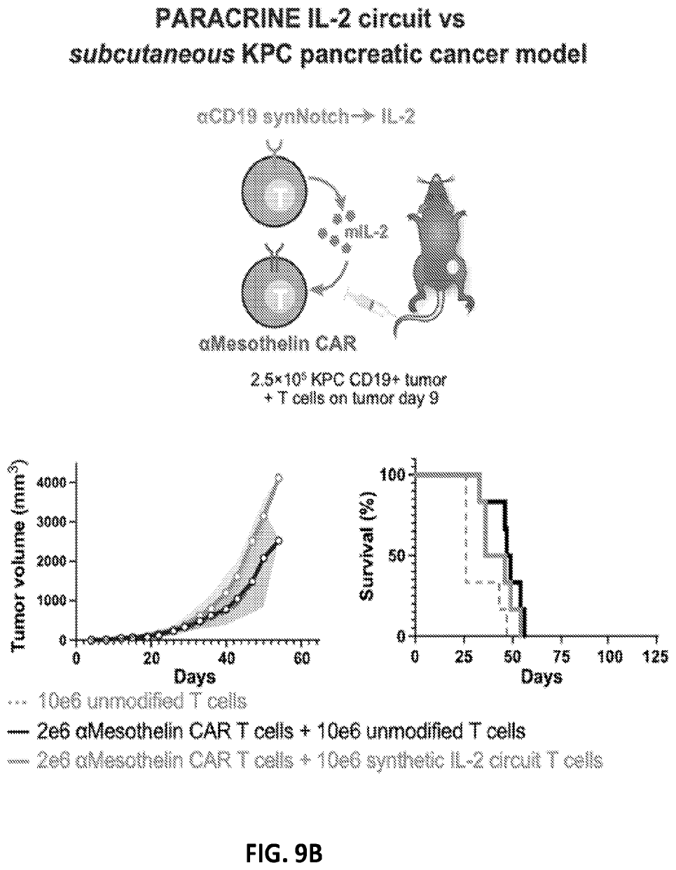

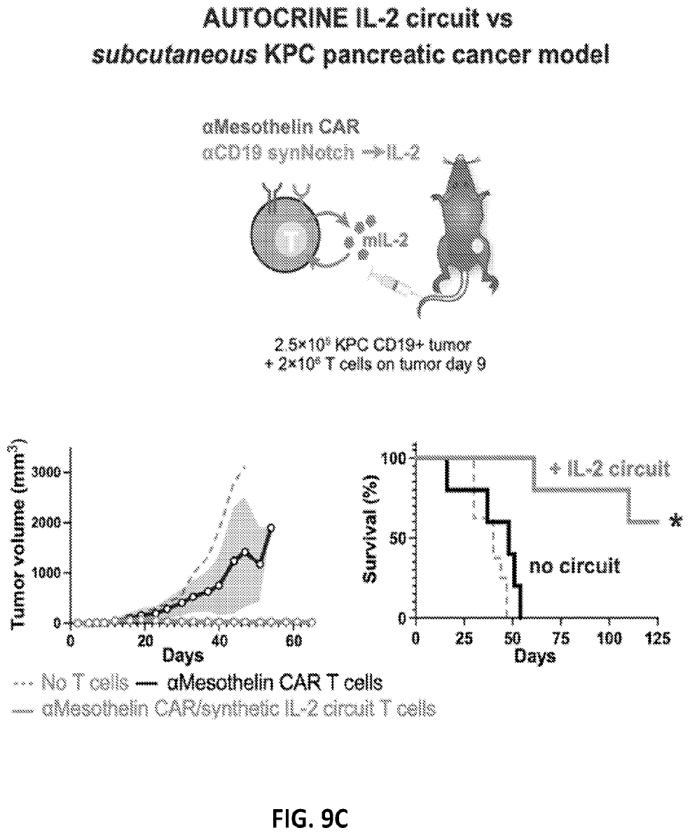

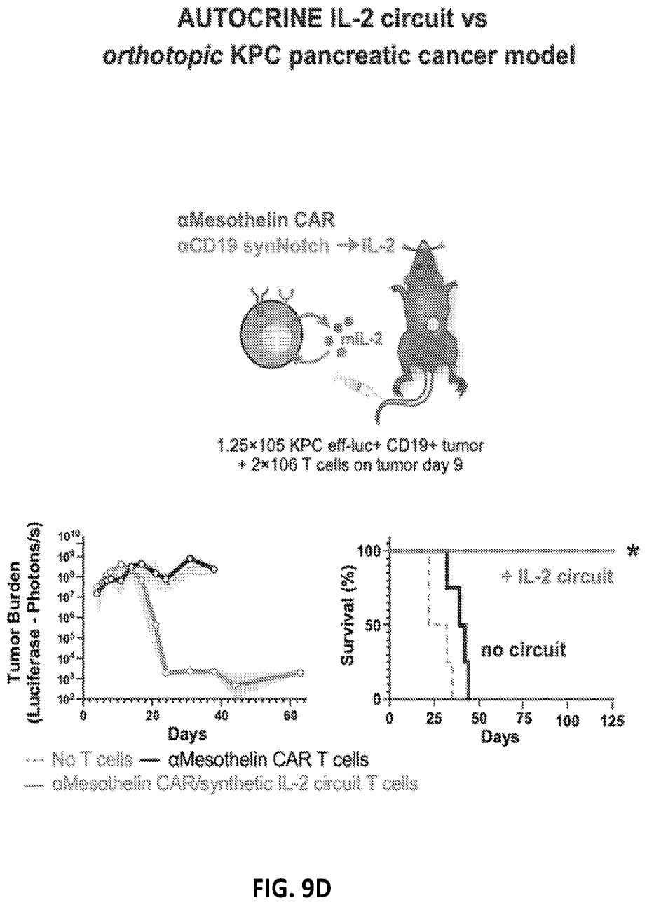

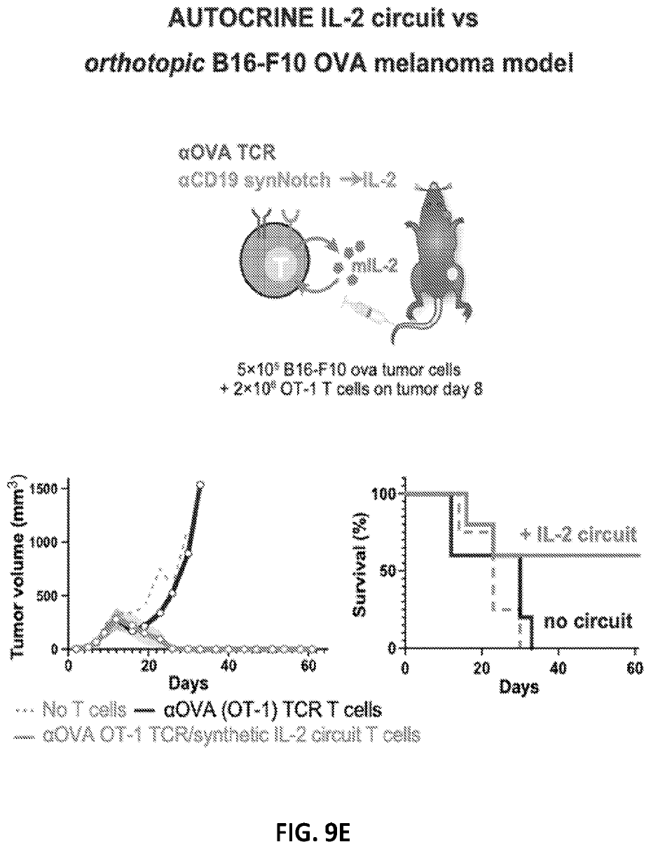

Although the above results show that synthetic synNotch→IL-2 circuits can significantly enhance T cell activity and expansion in immunodeficient mouse tumor models, we wanted to test whether they could also be effective in immunocompetent mouse models. Important factors influencing IL-2 production and consumption are likely missing in immunodeficient mouse models. Key missing factors include inhibitors of T cell activation (28) and the presence of competing IL-2 consumer cells (e.g. both native T cells, and T regulatory cells), which could significantly lower the effectiveness of synthetically produced IL-2 within tumors (29, 30). To study the effects of local IL-2 production within fully immunocompetent mouse tumor models, we rebuilt our synthetic IL-2 circuit in primary mouse T cells (FIG. 9A). Primary CD3+mouse T cells were engineered to express an anti-human-CD19 synNotch→mouse IL-2 (mIL-2) circuit. This circuit resulted in synNotch-induced proliferation of mouse T cells in vitro, just as was observed previously with human T cells (not shown).

We then chose to deploy this IL-2 circuit in targeting the mouse pancreatic tumor model KPC (KrasLSL.G12D/+; p53R172H/+; PdxCretg/+) (31, 32), as this immune-excluded tumor exhibits the challenging immunotherapy refractory features of pancreatic ductal adenocarcinoma (PDAC) (33). Like most pancreatic ductal adenocarcinomas, these cells express the tumor target antigen mesothelin (34). Although anti-mesothelin mouse CAR T cells show robust cytotoxicity against KPC cells in vitro (not shown), they show limited to no tumor control of KPC tumors in vivo (not shown). Thus, this immune competent mouse model replicates the poor in vivo therapeutic efficacy reported in early phase clinical trials of standard anti-mesothelin CAR T cells in pancreatic cancer (3), making it an ideal model in which to test enhancement of the CAR T cells with synthetic IL-2 circuits. We engineered KPC tumor cells that, in addition to endogenously expressing the CAR antigen (mesothelin), also expressed a model synNotch antigen (human CD19).

We first tested CAR T cell enhancement by a paracrine synNotch→mIL-2 circuit. Anti-mesothelin CAR T cells were co-injected with a second T cell population expressing the anti-CD19 synNotch→mIL-2 circuit. Distinct from our studies in immunodeficient mice, these paracrine IL-2 circuit cells failed to improve tumor control in an immune competent context (FIG. 9B). Instead, we found that in this fully immunocompetent tumor model, improved CAR T cell-mediated tumor control was only observed with the autocrine configuration of the synthetic IL-2 circuit—i.e. the cytotoxic receptor (CAR) and the synNotch→IL-2 circuit must be encapsulated within the same cell (FIG. 9C). We hypothesize that the presence of competing host IL-2 consumer cells (e.g. bystander T cells and Tregs) in immune-competent models contributes to this major difference between the autocrine and paracrine circuits (i.e. paracrine circuits might be more sensitive to competing IL-2 sink cells), a model consistent with more in depth tumor profiling data in later sections of this paper.

The autocrine synthetic IL-2 circuit anti-Mesothelin CAR-T cells were extremely potent. In an even more challenging immune-competent mouse model, in which KPC tumors were engrafted orthotopically in the pancreas, complete tumor clearance was observed upon treatment (FIG. 9D)—100% of mice survived, compared with 0% with CAR only T cells. Simply increasing the dose of anti-Mesothelin CAR-T cells had a negligible effect compared to addition of the synthetic IL-2 circuit (not shown).

This type of autocrine IL-2 circuit also shows similar dramatic therapeutic improvement in treating a different type of immune-excluded solid tumor—B16-F10 OVA intradermal melanoma tumors, treated with OT-1 TCR expressing T cells (FIG. 9E). Here again, OT-1 T cells without the cytokine circuit are ineffective in vivo in immune competent models (despite in vitro cytotoxic activity—not shown). Only when the OT-1 TCR is co-expressed with the autocrine synNotch→IL-2 circuit, do we observe effective infiltration and tumor clearance in the immune competent model.

### Comparison to Other Strategies of IL-2 Co-Delivery.

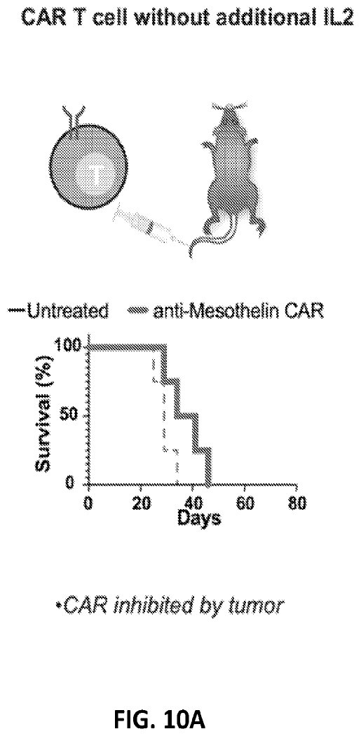

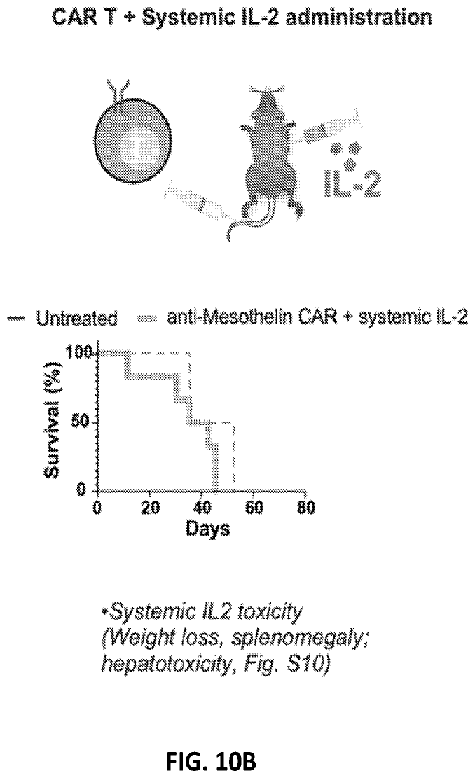

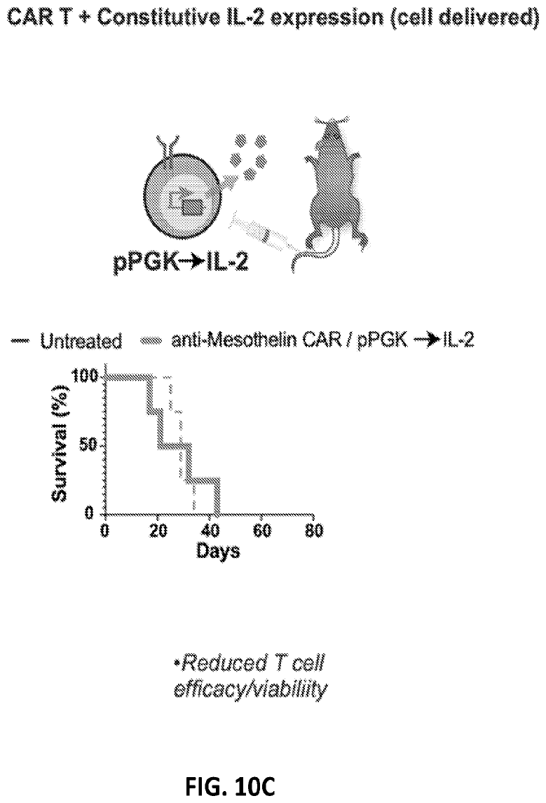

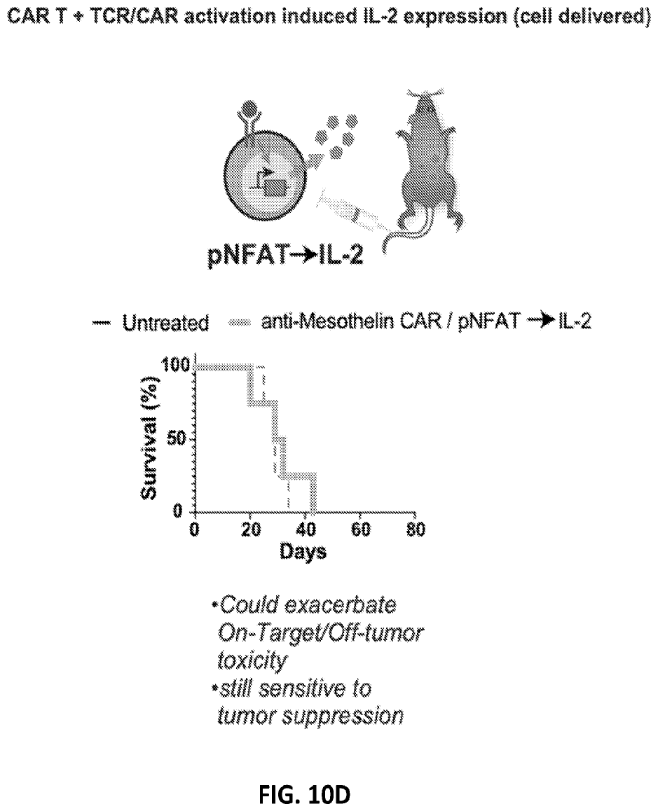

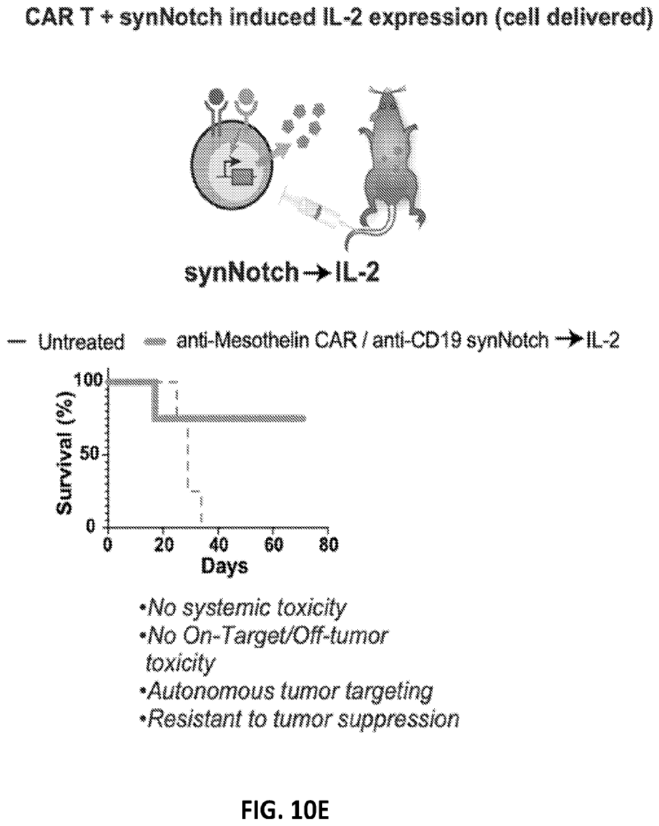

Importantly, this strong therapeutic improvement was not observed with other methods of co-delivering IL-2 with a CAR T cell. We tested systemic co-administration of IL-2 at maximum-tolerated doses (35) (FIG. 10B), expression of IL-2 in the CAR T cell from a constitutive promoter (“armored CAR”) (FIG. 10C), or expression of IL-2 from a T cell activated promoter such as pNFAT (36) (FIG. 10D).

Systemically injected IL-2 led to systemic toxicity without improving CAR T cell activity (not shown). Constitutive production of IL-2 was unable to support T cell proliferation in vivo (not shown) likely in part due to significant silencing of the constitutive IL-2 transgene (not shown) (37). IL-2 can have a biphasic effect on T cell survival (38) in part due to promotion of activation induced cell death (39) and T cell differentiation (40). We find that such negative effects are exacerbated by constitutive IL-2 production (not shown). This suggests that when and how the IL-2 cytokine is produced is critical in determining the outcome.

Importantly, despite its potent anti-tumor efficacy, the synNotch→IL-2 circuit showed no evidence of systemic cytokine toxicity or exacerbation of CAR T cell toxicity, as assessed by mouse survival, body weight, spleen weight, and measurements of hepatotoxicity (not shown). Moreover, the required recognition of two antigen inputs (CAR and synNotch antigens) should further enhance the specificity of tumor targeting (as seen by specific targeting to dual antigen tumor and reduced hepatotoxicity, not shown). In summary, combining a tumor-reactive TCR/CAR with an autocrine synNotch→IL-2 circuit, results in uniquely potent and localized anti-tumor enhancement.

Synthetic IL-2 Circuit Drives T Cell Infiltration into Immune Excluded Tumors

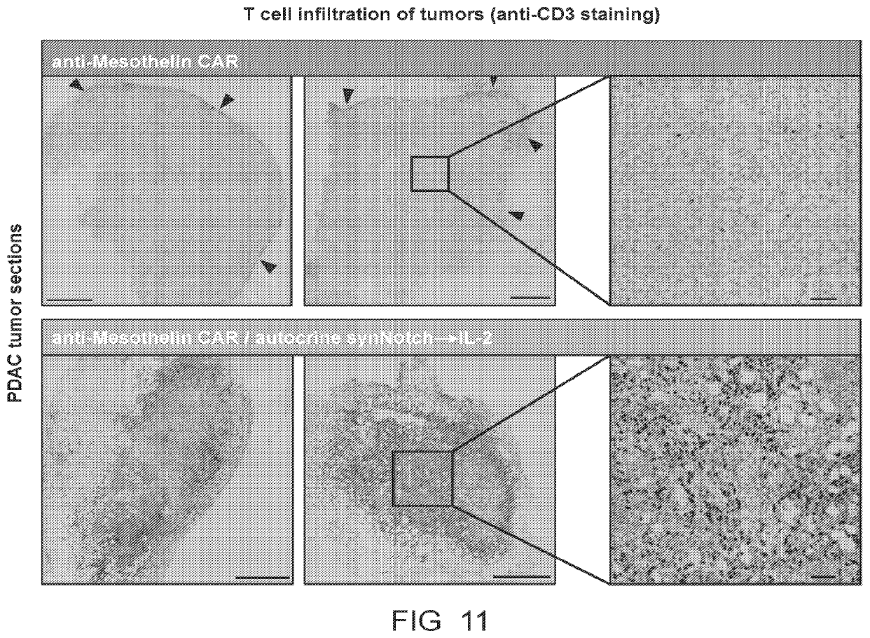

To better characterize how this autocrine synthetic IL-2 circuit improves CAR T cell control of syngeneic pancreatic tumor models, we profiled the tumors in more depth during treatment. We collected KPC pancreatic tumor specimens at the beginning and well into tumor regression (8 days and 23 days after T cell treatment) and measured CD3⁺ T cell infiltration using immunohistochemistry. Tumors treated with standard anti-mesothelin CAR T cells displayed a classic immune-excluded phenotype, with very limited T cell infiltrate inside the tumor core and most T cells gathered at the tumor periphery (FIG. 11, top). In contrast, tumors treated with CAR T cells containing the synthetic autocrine IL-2 circuit showed substantially increased infiltration of T cells throughout the tumor core (FIG. 11, bottom). A similar infiltration and expansion of the CD8+ lymphocytes also seen in B16-F10 OVA melanoma tumors sampled 10 days after treatment with OT-1 T cells bearing the synthetic IL-2 circuit (not shown).

To profile the tumors in more detail, we performed flow cytometry and CyTOF analyses on excised and dissociated tumors. To track the endogenous (host) T cells independently from the adoptively transferred CAR T cells, we adoptively transferred congenic Thy 1.1 or CD45.1 CAR T cells into Thy 1.2 or CD45.2 mice, respectively, allowing us to clearly distinguish endogenous from transplanted T cells by FACS.

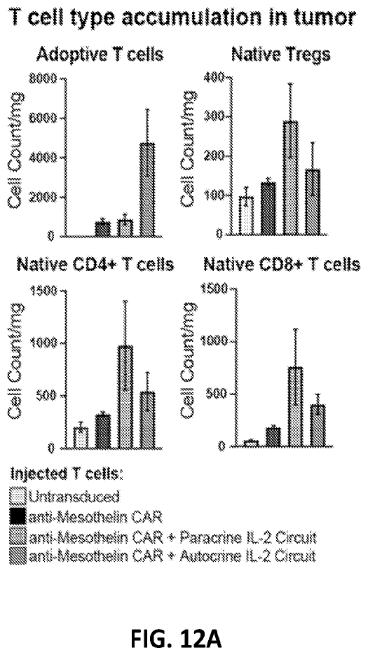

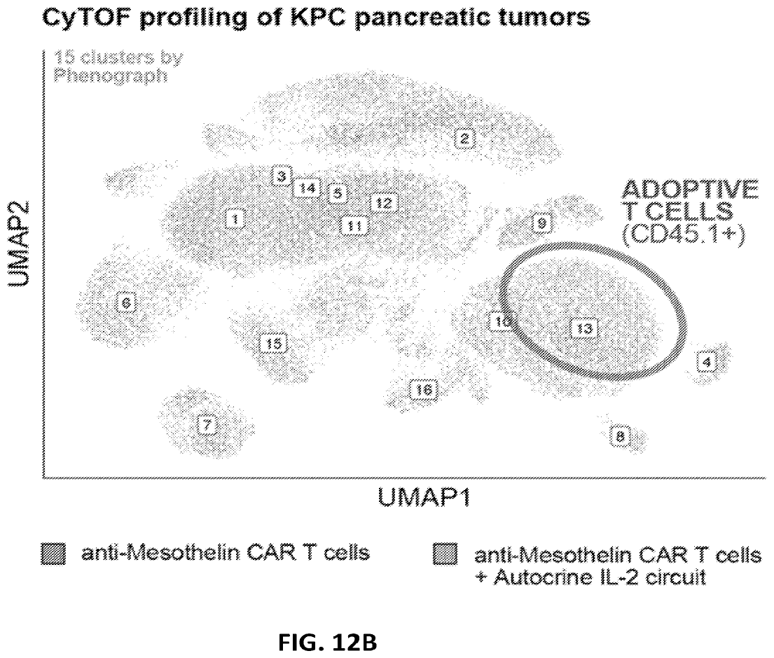

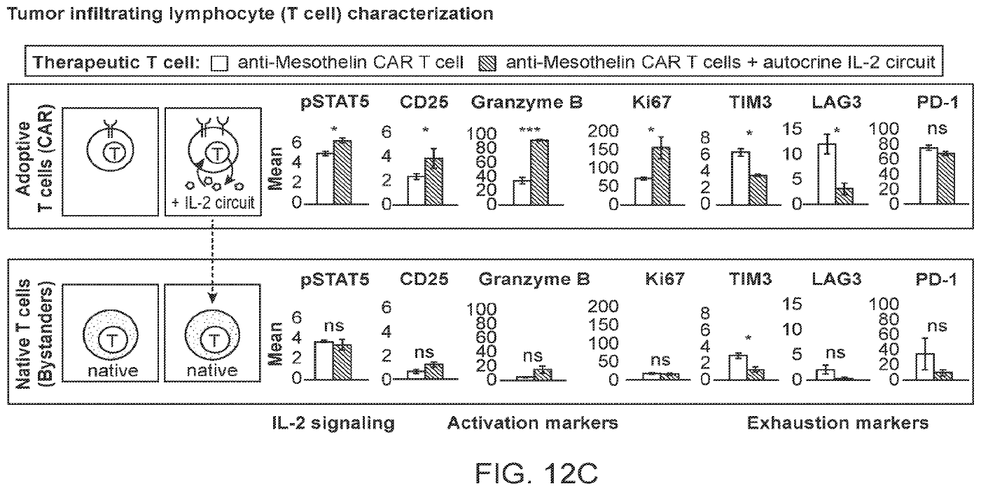

These studies showed that the engineered autocrine T cells (expressing both CAR and the synNotch→IL2 circuit) drove substantial intra-tumoral infiltration of both adoptively transferred (engineered) T cells and native host T cells (FIG. 12A). In contrast parallel analysis of tumors treated with the paracrine synNotch→IL-2 circuit (CAR and synthetic cytokine circuit are expressed by two separate, co-injected cell types) showed expansion of native T cells only and no expansion of the adoptively transferred CAR T cells (FIG. 12A), suggesting that in the paracrine configuration, induced IL-2 was primarily consumed by competing native T cells, leaving little available to drive expansion of the rarer CAR T cells.

Unsupervised clustering (41) of the CyTOF measurements (from the CD45⁺ immune cell infiltrate in KPC tumors) identified that the primary therapeutic effect of the autocrine IL-2 circuit was to enrich the population of activated adoptively transferred CAR T cells (FIG. 12B). Little change was seen in the myeloid compartments (42), suggesting that synthetic IL-2 production acts primarily to drive T cell infiltration (both native and adoptive) and not by altering myeloid cell associated immune suppression. Furthermore, the expansion of T cells was completely constrained to the tumor-no changes were seen in immune cells from isolated spleens by flow cytometry or CyTOF analysis (not shown), highlighting the focused local activity of the engineered cytokine circuit.

In addition to driving expansion of cytotoxic T cells in these immunologically cold tumors, the synthetic autocrine IL-2 circuit improved the phenotypes of the CAR T cells that infiltrate the tumor. CyTOF analysis showed that the synthetic autocrine IL-2 circuit upregulated markers of T cell activation (CD25), effector activity (Granzyme B) and proliferation (Ki67). Conversely, these IL-2 enhanced T cells also showed reduced expression of markers of exhaustion (Tim3, Lag3, PD-1) (FIG. 12C) (43). Most native T cells (non-CAR) found in the tumors, however, appear to act simply as IL-2 sinks-they did not show markers of activation, effector function, proliferation, or exhaustion (FIG. 12C), but instead largely exhibited a naïve phenotype (not shown). The phenotype of the regulatory T cell population was mostly unchanged (not shown). These findings suggest that the tumor has a significant population of native host T cells that, in bulk, compete to consume IL-2 without contributing to the anti-tumor response, (akin to Treg suppression via IL-2 consumption).

### DISCUSSION

Cell Delivered IL-2 is a Powerful Tool to Synergize with Therapeutic T Cells

Cytokines such as IL-2 have long been known as powerful stimulators of anti-tumor immunity (44). However, systemic IL-2 delivery is also well known to be highly toxic, leading to a broad set of adverse effects including capillary leak syndrome, thereby greatly limiting its therapeutic use (45). Most current efforts in IL-2 engineering have focused on engineering the cytokine to be more selective for a tumor. Here instead we use a different strategy: harnessing the power of an engineered cell to identify a tumor and locally deliver IL-2 exactly where it is needed. We show that cell-mediated local cytokine (IL-2) delivery can effectively overcome immune suppression, augmenting CAR T cells to efficiently clear multiple immune-excluded tumor models (pancreatic cancer and melanoma) that are otherwise nearly completely resistant to standard CAR T cell treatment.

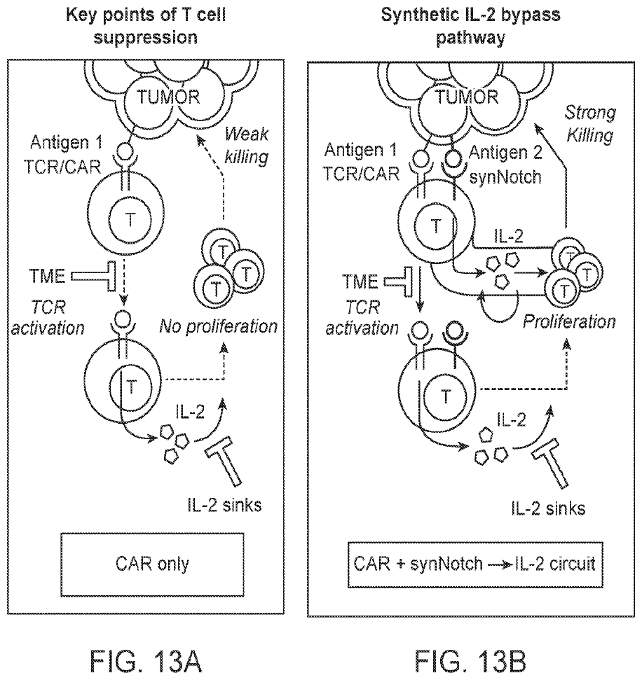

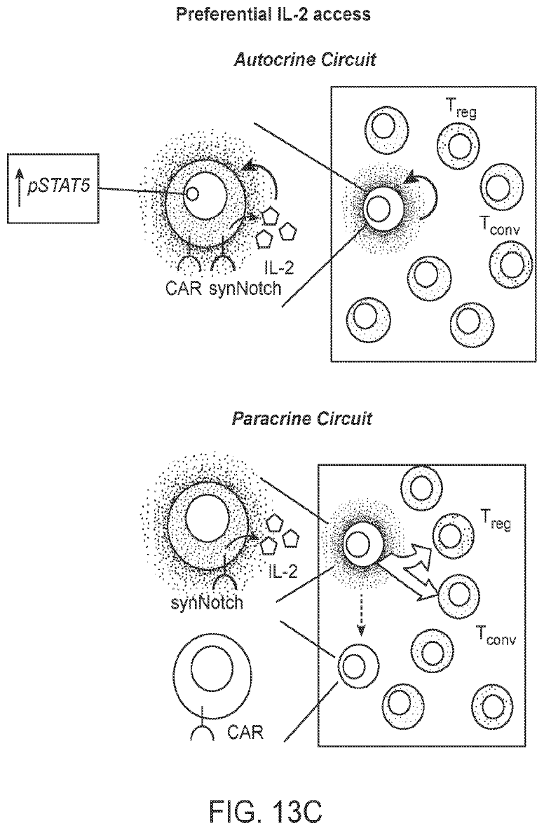

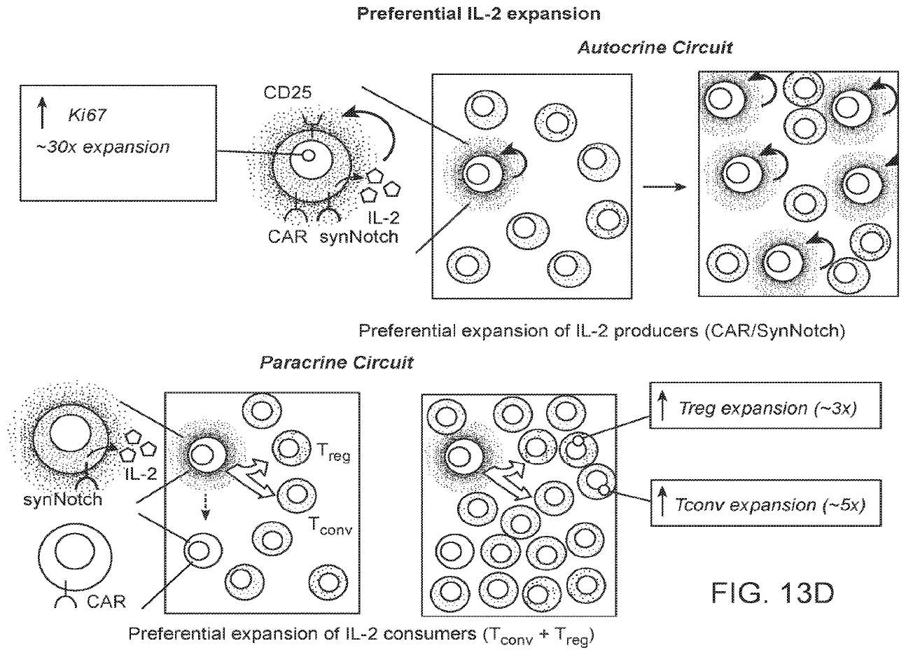

However, the exact manner of by which the cytokine is produced is important to its success. First, cytokine production must be dynamically regulated (inducible). Constant production of IL-2 risks exacerbating off-target toxicity. Moreover, constitutive IL-2 expression in T cells has negative effects—it leads to terminal differentiation, fails to drive autonomous proliferation, and is limited by payload silencing. Second, in order to bypass TCR/CAR suppression by the tumor microenvironment, induction of IL-2 production should be independent of the TCR activation pathway (e.g. NFAT promoter induced IL-2 still requires TCR/CAR activation to be triggered). We find that one powerful solution to this constraint is to engineer a synthetic signal transduction pathway that is tumor-triggered, but bypasses the native CAR/TCR activation pathway (FIG. 13A,B). Using a synNotch receptor that detects the tumor to drive IL-2 production provides a simple and modular way to achieve this goal. The synNotch IL-2 circuit can maintain payload expression in spite of T cell inhibition or exhaustion (not shown).

Finally, we find that simply having an immune cell that can individually produce high levels of IL-2 in the tumor is not sufficient to overcome suppression. The specific circuit architecture is important, including exactly which cells produce IL-2. We find that an effective therapeutic response is only observed with an autocrine IL-2 circuit (i.e. synthetic IL-2 induction pathway is contained within the same cell as the anti-tumor CAR/TCR).

### Mechanisms Underlying Autocrine/Paracrine Circuit Differences

It is likely that there are multiple mechanisms that contribute to the far better efficacy of the autocrine circuit (FIG. 13C, D). These mechanisms are tightly interlinked, and likely act in a highly cooperative manner, thus making it difficult to precisely pinpoint the relative contribution of each mechanism.

First, it is likely that autocrine cells have preferential access to self-produced IL-2, especially in environments with competing IL-2 sinks. Paracrine circuits must physically transfer IL-2 further through space from a producer T cell to an effector T cell. This becomes challenging in the presence of competing IL-2 consumer cells (e.g. Tregs in immune competent models), which can greatly reduce the effective length scale of IL-2 signaling creating gradients that drop off sharply around IL-2 sources (46). Here, in both the autocrine and paracrine circuit, we observe an expansion of host Treg cells (FIG. 13A); however, we also see a much larger expansion of naïve T cells (not shown). These results suggest that host conventional T cells in the tumor can also play a significant role as IL-2 sinks, especially given their vast excess population. Although it is difficult to parse out the relative contribution of these Tregs VS conventional T cells as IL-2 consumers, it is not uncommon to observe the presence of large numbers of tumor infiltrating but non tumor-reactive T cells (30). Whatever their relative contribution, these IL-2 consumers are both expected to decrease the effective signaling distance of IL-2 producers (47, 48), which would strongly favor the efficacy of autocrine over paracrine IL-2 production in driving CAR T cell expansion.

Second, it also likely that autocrine cells are capable of preferential expansion in response to the available pool of IL-2. There is a unique proliferative positive feedback loop that could in principle take place with T cells that can induce both IL-2 and TCR/CAR activation. T cell activation can both trigger an initially IL-2 independent proliferative response (49) as well as induce expression of the high affinity IL-2 receptor subunit, CD25 (not shown), which allows T cells to outcompete other T cells for available IL-2. Because an autocrine circuit cell contains both the CAR and synNotch→IL-2 circuit, it has the capability to become both a preferred IL-2 responder (via T cell activation) and strong IL-2 producer (via synNotch activation) within a tumor. We hypothesize that these dually activated autocrine cells could thereby initiate a powerful population level positive feedback loop that builds up even higher levels of intra-tumoral IL-2 due to preferential expansion of better IL-2 consumers/responders. This population positive feedback would not take place in the paracrine circuit, as the IL-2 producers do not upregulate CD25 (not shown) and their IL-2 production would largely contribute to expanding competing T cells (such as Tregs) that act to suppress T cell based immunity. Several pieces of evidence support this model of preferred expansion of autocrine circuit cells. First, only in the autocrine CAR T cells, do we observe significantly higher expression of the proliferation marker Ki67 (FIG. 13C). Second, we notably do not see increased tumor control with an autocrine circuit that produces the homeostatic cytokine IL-7 (not shown). Further experiments will be needed to definitively evaluate the relative contributions of the multiple mechanisms discussed in this model.

### Essential Requirements to Bypass Tumor Immunosuppression

Our efforts to systematically design CAR T circuits that couple IL-2 production/signaling with CAR signaling in alternative ways also sheds light onto the basic design principles of native T cell activation. The T cell system has evolved to severely restrict improper activation, but at the same time to be able to launch a locally explosive response, once triggered. Population-level positive feedback signaling using a shared cytokine (IL-2) allows this type of digital response between on and off states (50, 51). In this model, T cells should not only be stimulated by the proper antigens, but they should also subsequently produce enough IL-2 to overcome the threshold set by competing IL-2 consumer cells present throughout the microenvironment (52). This control mechanism, however, provides weak points that tumors can take advantage of for immune suppression. Many tumors keep a strong T cell response in check, either by blocking T cell activation (28), or increasing competition for amplification factors like IL-2.

Here we show that it is possible to still reconstitute the pathways required for a strong anti-tumor T cells response (i.e., rewiring the cell such that T cell activation, co-stimulation and IL-2 signaling are still cooperatively stimulated), but in a way that now evades the major tumor suppressive mechanisms. Normally IL-2 is produced after T Cell activation and acts as a critical amplifier of T cell activity. By placing IL-2 production under the control of a new TCR-independent but still tumor-targeted synthetic receptor we can now produce IL-2 immediately and consistently after tumor entry despite suppression of T cell activation. In addition, normally IL-2 consumers apply a selective pressure only allowing strongly activated effector T cells to expand (52). By coupling TCR/CAR activation and synNotch driven IL-2 production in an autocrine IL-2 circuit we can selectively expand the engineered therapeutic T cell population out of a background of competing IL-2 consumers. These rewired cells ultimately activate the same critical pathways (TCR and IL-2 pathways) as seen in native T cell responses but do so in a different temporal order and in response to different inputs allowing them to be far more effective as a tumor-targeted therapy (FIG. 13A-D). The engineered circuit maintains the explosive cell expansion necessary for a robust anti-tumor activity but triggered in a manner that evades the major mechanisms of immunosuppression.

### The Power of Alternatively Wired Immune Cell Circuits.

In summary, we have been able to use flexible synthetic biology tools, such as the synNotch receptor system, to create new, alternative ways to rapidly establish both the TCR and IL-2 pathway activity required for an effective and sustained T cell response. The resulting bypass channel for IL-2 production allows for improved tumor control and reduced toxicity compared to alternative mechanisms of IL-2 delivery. Synthetic cytokine production circuits may represent a general solution for engineering immune cell therapies that can function more effectively in hostile tumor microenvironments, illustrating the power of customizing immune responses in highly precise but novel ways.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An engineered immune cell comprising the following components:
(a) a nucleic acid encoding an immune receptor that is activated by binding to a cancer-associated antigen in a solid tumor;
(b) a binding triggered transcriptional switch (BTTS) that is independently activated by either a tissue- or a cancer-associated antigen in the solid tumor; and
(c) a nucleic acid encoding a pro-inflammatory protein,
wherein:
binding of the immune receptor to the cancer-associated antigen activates the immune cell; and
binding of the BTTS to its antigen activates expression of the pro-inflammatory protein and, optionally, the immune receptor if the immune receptor is not constitutively expressed in the cell.

2. The engineered immune cell of claim 1, wherein the immune receptor is a chimeric antigen receptor (CAR) or an T cell receptor (TCR).

3. The engineered immune cell of claim 1, wherein the immune receptor and the BTTS recognize the same antigen.

4. The engineered immune cell of claim 1, wherein the immune receptor and the BTTS recognize different antigens.

5. The engineered immune cell of claim 1, wherein the pro-inflammatory protein is a pro-inflammatory cytokine.

6. The engineered immune cell of claim 1, wherein the pro-inflammatory protein is a cytokine selected from IL-2, IL-12, IL-15, IL-7, CD40L, or a non-natural variant of IL-2, an orthogonal cytokine, IL-12, IL-15, IL-7, CD40L that has pro-inflammatory activity, an immune checkpoint inhibitor, a decoy resistant IL-18, a dominant negative TGF-β, a dominant negative TGFb receptor, or a TGFb inhibitor/agonist.

7. The engineered immune cell of any prier claim 1, the solid tumor is a tumor of lung cancer, colorectal cancer, pancreatic cancer, prostate cancer, liver and biliary tract cancers, bladder cancer, brain cancer (e.g., GBM), esophageal cancer, ovarian cancer, kidney cancer, melanoma, gastric/stomach cancer, breast cancer, mesothelioma, uterine, testicular, and head and neck (including thyroid).

8. The engineered immune cell of claim 1, wherein cancer-associated antigen recognized by the immune receptor is selected from Table 1.

9. The engineered immune cell of claim 1, wherein the antigen recognized by the BTTS is tissue specific or selected from Table 1.

10. The engineered immune cell of claim 1, wherein the BTTS comprises one or more protease cleavage sites and an intracellular domain comprising a transcriptional activator, wherein binding of the BTTS to the tissue- or a cancer-associated antigen on another cell causes the BTTS to be cleaved at the protease cleavage site, thereby releasing the transcriptional activator, and wherein the released transcriptional activator induces expression of the pro-inflammatory protein.

11. The engineered immune cell of claim 10, wherein the one or more protease cleavage sites comprises a cleavage site for a γ-secretase

12. The engineered immune cell of claim 1, wherein the BTTS comprises:
i. an extracellular binding domain that binds to the tissue- or a cancer-associated antigen in the solid tumor,
ii. a force sensing region,
iii. a transmembrane domain,
iv. one or more force-dependent cleavage sites that are cleaved when the force sensing region is activated, and
v. an intracellular domain comprising a transcriptional activator, where binding of the extracellular binding domain to the tissue- or a cancer-associated antigen induces proteolytic cleavage of the one or more force-dependent cleavage sites to release the transcriptional activator,
wherein the transcriptional activator induces expression of the pro-inflammatory protein and the immune receptor, if the immune receptor is not constitutively expressed.

13. The engineered immune cell of claim 1, wherein the BTTS comprises an extracellular domain that comprises a scFv or nanobody that binds to the tissue- or cancer-associated antigen.

14. The engineered immune cell of claim 1, wherein the immune cell is a myeloid or lymphoid cell.

15. The engineered immune cell of claim 1, wherein the immune cell a T lymphocyte, a B lymphocyte, macrophage or a Natural Killer cell.

16. The engineered immune cell of claim 1, wherein expression of the immune receptor is constitutive in the cell.

17. The engineered immune cell of claim 1, wherein binding of the BTTS to its antigen activates expression of the pro-inflammatory protein and the immune receptor in the cell.

18. A method of treating a subject for a solid tumor, comprising:
administering to the subject a cell of claim 1.

19. The method of claim 18, wherein:
(a) the subject has a cancer selected from the cancers listed in Table 2;
(b) the immune receptor is activated by binding to an antigen associated with the cancer in Table 2; and
(c) the BTTS is independently activated by binding to either a tissue-specific antigen for the cancer or to an antigen associated with the cancer in Table 2; wherein:
binding of the immune receptor to its antigen activates the immune cell; and
binding of the BTTS to its antigen activates expression of the pro-inflammatory protein and, optionally, the immune receptor if the immune receptor is not constitutively expressed in the cell.

20. The method of claim 18, wherein the subject has lung cancer, colorectal cancer, pancreatic cancer, prostate cancer, liver and/or biliary tract cancer, bladder cancer, brain cancer (e.g., GBM), esophageal cancer, ovarian cancer, kidney cancer, melanoma, gastric/stomach cancer, breast cancer, mesothelioma, uterine cancer, testicular cancer, or head and neck (including thyroid) cancer.
